(19) United States
(12) Patent Application Publication
(10) Pub. No.: US 2016/0109420 A1
(43) Pub. Date: Apr. 21, 2016

(54) FAILURE DETERMINATION DEVICE FOR EMISSION CONTROL APPARATUS OF INTERNAL COMBUSTION ENGINE

(71) Applicant: Toyota Jidosha Kabushiki Kaisha, Toyota-shi Aichi-ken (JP)

(72) Inventors: Kenji Furui, Sunto-gun Shizuoka-ken (JP); Toru Kidokoro, Hadano-shi Kanagawa-ken (JP); Taiga Hagimoto, Susono-shi Shizuoka-ken (JP); Arifumi Matsumoto, Gotenba-shi Shizuoka-ken (JP); Akifumi Uozumi, Susono-shi Shizuoka-ken (JP)

(21) Appl. No.: 14/882,807

(22) Filed: Oct. 14, 2015

(30) Foreign Application Priority Data
Oct. 17, 2014 (JP) 2014-213048

(51) Int. Cl. G01N 33/00

(52) U.S. Cl. CPC G01N 33/0037; G01N 33/0073

(57) ABSTRACT

When individually controlling a plurality of supply valves is not available, an object of the invention is to determine which of the plurality of supply valves is abnormal with high accuracy, while suppressing a cost increase. A first supply valve, a first selective reduction NOx catalyst, a second supply valve, a second selective reduction NOx catalyst and a NOx sensor are sequentially provided in an exhaust conduit. With a view to identifying which of abnormality of the first supply valve and abnormality of the second supply valve, an instruction is given to the first supply valve and the second supply valve to increase a supply amount of a reducing agent. This identification is based on a NOx concentration detected by the NOx sensor after elapse of a first specified time duration since an instruction time point that is a time point when this instruction is given.

### CROSS REFERENCE TO RELATED APPLICATIONS

This application claims priority to Japanese Patent Application No. 2014-213048 filed on Oct. 17, 2014, the entire contents of which are incorporated by reference herein.

### BACKGROUND

1. Technical Field

The present invention relates to a failure determination device for an emission control apparatus of an internal combustion engine.

2. Description of the Related Art

A known selective reduction NOx catalyst (hereinafter simply referred to as “NOx catalyst”) uses ammonia as a reducing agent to convert NOx included in exhaust emission from an internal combustion engine. A supply valve or the like is located upstream of this NOx catalyst to supply ammonia or an ammonia precursor into the exhaust emission. The precursor of ammonia is, for example, urea. In the description below, ammonia and the precursor of ammonia are collectively called “reducing agent”.

One proposed technique computes a model simulating a pressure change in a reducing agent conduit during supply control or after supply control of the reducing agent and compares the computed model with a stored model to determine whether abnormality occurs in a reducing agent supply device (see, for example, Patent Literature 1).

### CITATION LIST

### Patent Literature

PTL 1: JP 2010-174786A

PTL 2: JP 2010-270614A

### SUMMARY

In order to enhance the NOx conversion rate, one possible configuration may include two NOx catalysts arranged in series in an exhaust conduit. Additionally, supply valves may be provided upstream of the respective NOx catalysts to supply the reducing agent. In this configuration, individually controlling the respective supply valves is likely to complicate the control. The control may be simplified, on the other hand, by controlling the respective supply valves together. When individually controlling the supply valves is not available, however, it is difficult to identify which of the supply valves is abnormal in the case of abnormality of one of the supply valves. No matter which of the supply valves is abnormal, the NOx conversion rate of the entire system is similarly decreased. It is accordingly difficult to identify which of the supply valves is abnormal, based on the NOx conversion rate. NOx sensors may be provided downstream of the respective NOx catalysts to calculate the NOx conversion rates in the respective NOx catalysts. This configuration allows for identification of which of the supply valves is abnormal. Providing a plurality of NOx sensors, however, undesirably increases the cost.

By taking into account the problems described above, when individually controlling a plurality of supply valves is not available, an object of the invention is to identify which of the plurality of supply valves is abnormal with high accuracy, while suppressing a cost increase.

In order to solve the above problems, according to one aspect of the invention, there is provided a failure determination device for an emission control apparatus of an internal combustion engine. The failure determination device comprises a first supply valve that is provided in an exhaust conduit of the internal combustion engine to supply a reducing agent into the exhaust conduit; a first selective reduction NOx catalyst that is provided downstream of the first supply valve in the exhaust conduit to selectively reduce NOx with the reducing agent adsorbed to the first selective reduction NOx catalyst; a second supply valve that is provided downstream of the first selective reduction NOx catalyst in the exhaust conduit to supply the reducing agent into the exhaust conduit; a second selective reduction NOx catalyst that is provided downstream of the second supply valve in the exhaust conduit to reduce NOx with the reducing agent adsorbed to the second selective reduction NOx catalyst; a NOx sensor that is configured to detect a NOx concentration in an exhaust emission flowing out of the second selective reduction NOx catalyst; and a controller that is configured to determine a supply amount of the reducing agent based on an amount of NOx flowing into the first selective reduction NOx catalyst and give an identical instruction to operate the first supply valve and the second supply valve. The controller gives an instruction to the first supply valve and the second supply valve such as to make a supply amount of the reducing agent from the first supply valve and the second supply valve larger than the supply amount of the reducing agent determined based on the amount of NOx flowing into the first selective reduction NOx catalyst, and determines whether the first supply valve is abnormal or the second supply valve is abnormal, based on a NOx concentration detected by the NOx sensor after elapse of a first specified time duration since a certain instruction time point when the instruction is given.

Abnormality of the first supply valve or the second supply valve includes the case where no reducing agent is supplied from one of the first supply valve and the second supply valve and the case where the reducing agent is supplied from one of the first supply valve and the second supply valve at such a level that hardly contributes to conversion of NOx. The first supply valve and the second supply valve are configured to supply, for example, urea water or ammonia as the reducing agent into exhaust emission.

In the case of operation of the first supply valve and the second supply valve to perform control that supplies an amount of reducing agent from the first supply valve and the second supply valve according to the amount of NOx discharged from the internal combustion engine (hereinafter called regular control), a NOx conversion rate of the entire system is decreased, irrespective of which of the supply valves is abnormal. The supply amount of the reducing agent in the regular control is determined such as to provide a NOx conversion rate within a target range, based on the amounts of the reducing agent adsorbed to the first selective reduction NOx catalyst (also called first NOx catalyst) and to the second selective reduction NOx catalyst (also called second NOx catalyst) in the case where both the first supply valve and the second supply valve are normal. The NOx conversion rate is calculated from the NOx concentration in the exhaust emission flowing into the first NOx catalyst and the NOx concentration detected by the NOx sensor.

In the failure determination device of this aspect, in order to determine whether the first supply valve is abnormal or the second supply valve is abnormal, the controller gives an instruction to the first supply valve and the second supply valve to supply a larger amount of the reducing agent than the supply amount in the regular control. In the description below, the supply amount of the reducing agent increased from the supply amount in the regular control is called criterion supply amount.

In the case of abnormality of the second supply valve, the first supply valve supplies the reducing agent to the first NOx catalyst, so that the first NOx catalyst works to convert NOx. In this state, the larger amount of the reducing agent than the amount in the regular control is supplied from the first supply valve. Part of the reducing agent supplied from the first supply valve accordingly flows out of the first NOx catalyst. The flow-out reducing agent is supplied to the second NOx catalyst, which accordingly allows for conversion of NOx.

In the case of abnormality of the first supply valve, on the other hand, an excess amount of the reducing agent is supplied from the second supply valve to the second NOx catalyst, so that the reducing agent flows out of the second NOx catalyst. The NOx sensor detects ammonia other than NOx. The presence of ammonia in the exhaust emission thus increases the detection value of the NOx sensor. This results in decreasing the NOx conversion rate calculated based on the detection value of the NOx sensor. Accordingly, in the case where the first supply valve is abnormal, the NOx conversion rate of the entire system is relatively low even after elapse of a certain time duration.

As described above, after elapse of the first specified time duration since the instruction time point, a difference is made between the NOx conversion rates in the case of abnormality of the first supply valve and in the case of abnormality of the second supply valve. It is thus determinable which of the first supply valve and the second supply valve is abnormal, based on the NOx conversion rate at this moment.

The first specified time duration may be a time duration that causes a difference between the NOx conversion rates in the case of abnormality of the first supply valve and in the case of abnormality of the second supply valve, since the instruction time point. For example, the first specified time duration may be a time duration that causes the reducing agent to achieve equilibrium in the second NOx catalyst in the case where the second supply valve is abnormal, since the instruction time point. The state that the reducing agent achieves equilibrium means the state that the amount of the reducing agent adsorbed to the catalyst is equivalent to the amount of the reducing agent released from the catalyst and the state that supply of the reducing agent to the catalyst does not increase the amount of the reducing agent adsorbed to the catalyst. In other words, the first specified time duration may be a time duration that causes a sufficient amount of the reducing agent to be adsorbed to the second NOx catalyst even in the case where the second supply valve is abnormal.

In the failure determination device for the emission control apparatus of the internal combustion engine according to the above aspect, the controller may determine abnormality of the second supply valve, when a NOx conversion rate calculated from the NOx concentration detected by the NOx sensor after elapse of the first specified time duration since the instruction time point is equal to or higher than a supply valve threshold. The controller may determine abnormality of the first supply valve, when the NOx conversion rate is lower than the supply valve threshold.

The supply valve threshold is a lower limit value of the NOx conversion rate in the case where the entire system is normal. The NOx conversion rate calculated from the NOx concentration detected by the NOx sensor denotes the NOx conversion rate of the entire system. Even in the case of abnormality of the second supply valve, giving an instruction to make the supply amount of the reducing agent equal to the criterion supply amount causes the reducing agent to be supplied from the first supply valve to the second NOx catalyst. This causes the NOx conversion rate of the entire system to be equal to or higher than the supply valve threshold. In the case of abnormality of the first supply valve, on the other hand, the first NOx catalyst fails to convert NOx, so that the NOx conversion rate is lower than the supply valve threshold. The first specified time duration may thus be a time duration that causes the reducing agent to achieve equilibrium in the second NOx catalyst when the second supply valve is abnormal, since the instruction time point as described above.

In the failure determination device for the emission control apparatus of the internal combustion engine according to the above aspect, the controller may determine occurrence of slight concentration abnormality that provides a low concentration of the reducing agent, when a NOx conversion rate calculated from the NOx concentration detected by the NOx sensor after elapse of a second specified time duration, which is a shorter time period than the first specified time duration, since the instruction time point is equal to or higher than a supply valve threshold. The controller may determine abnormality of either one of the first supply valve and the second supply valve, when the NOx conversion rate is lower than the supply valve threshold.

The slight concentration abnormality is abnormality of the concentration of the reducing agent and provides a lower concentration of the reducing agent than the concentration in the normal state. The slight concentration abnormality herein denotes such abnormality of the concentration of the reducing agent that causes the NOx conversion rate in the case of abnormality of the concentration of the reducing agent to be equivalent to or higher than the NOx conversion rate in the case of abnormality of either one of the first supply valve and the second supply valve in the regular control. In other words, the slight concentration abnormality denotes a relatively low degree of abnormality of the concentration of the reducing agent. The supply amount of the reducing agent in the case of slight concentration abnormality is equivalent to or higher than the supply amount of the reducing agent in the case of abnormality of either one of the first supply valve and the second supply valve. Accordingly, the case where the concentration of the reducing agent is zero and the case where the concentration of the reducing agent is not zero but is substantially close to zero are to be excluded from the slight concentration abnormality.

In the case of slight concentration abnormality, a low concentration of the reducing agent is supplied from the first supply valve and the second supply valve. Even when the reducing agent has a low concentration, increasing the supply amount of the reducing agent to the criterion supply amount causes a certain amount of the reducing agent to be eventually supplied to the first NOx catalyst and the second NOx catalyst. Accordingly, even in the case of slight concentration abnormality, the NOx conversion rate of the entire system may eventually become equal to or higher than the supply valve threshold.

The second specified time duration is a time duration that causes a difference between the NOx conversion rates in the case of abnormality of the second supply valve and in the case of slight concentration abnormality, since the instruction time point. For example, the second specified time duration may be a time duration that causes the reducing agent to achieve equilibrium in the first NOx catalyst when the first supply valve is normal, since the instruction time point. In this case, it is determinable whether the concentration of the reducing agent is abnormal by comparison between the NOx conversion rate after elapse of the second specified time duration and the supply valve threshold. This time duration may be approximately equal to a time duration that causes the reducing agent to achieve equilibrium in the second NOx catalyst when the second supply valve is normal. In the case of abnormality of the second supply valve, after elapse of the second specified time duration, only a small amount of the reducing agent is supplied to the second NOx catalyst. The NOx conversion rate is thus kept low. In the case of slight concentration abnormality, on the other hand, after elapse of the second specified time duration, a large amount of the reducing agent is supplied to the first NOx catalyst and the second NOx catalyst. The NOx conversion rate is thus increased.

In the failure determination device for the emission control apparatus of the internal combustion engine according to the above aspect, the controller may determine occurrence of one of the slight concentration abnormality, abnormality of the first supply valve and abnormality of the second supply valve, when the NOx conversion rate calculated from the NOx concentration detected by the NOx sensor prior to the instruction time point is equal to or higher than a severe reducing agent abnormality threshold, which is a smaller threshold value than the supply valve threshold. The controller may determine occurrence of severe reducing agent abnormality that provides a lower concentration of the reducing agent than the concentration provided by the slight concentration abnormality, when the NOx conversion rate is lower than the severe reducing agent abnormality threshold.

The time prior to the instruction time point denotes the time when the regular control is performed. The severe reducing agent abnormality threshold is smaller than the supply valve threshold and may be a NOx conversion rate in the case of abnormality of either one of the first supply valve and the second supply valve in the regular control. Accordingly, when the NOx conversion rate in the regular control is equal to or higher than the severe reducing agent abnormality threshold, it is expected that one of the first supply valve and the second supply valve is normal. In the case of slight concentration abnormality, the NOx conversion rate is also equal to or higher than the severe reducing agent abnormality threshold.

When the NOx conversion rate is lower than the severe reducing agent abnormality threshold, on the other hand, the abnormality causes the concentration of the reducing agent to be lower than the concentration in the case of slight concentration abnormality. The case where the NOx conversion rate is lower than the severe reducing agent abnormality threshold includes the case where the concentration of the reducing agent is not zero but is low and the case where no reducing agent is present. The concentration of the reducing agent in this case may be, for example, such a concentration that causes the NOx conversion rate after elapse of the second specified time duration since the instruction time point to be lower than the supply valve threshold. The case where no reducing agent is present includes, for example, the case where the reducing agent is used up and the case where the concentration of the reducing agent is 0% (in the case of water or another liquid).

The failure determination device for the emission control apparatus of the internal combustion engine according to the above aspect may further comprise a reducing agent concentration sensor that is configured to detect a concentration of the reducing agent. The controller may determine occurrence of severe supply valve deterioration that is deterioration of both the first supply valve and the second supply valve, when the concentration of the reducing agent detected by the reducing agent concentration sensor is normal and a NOx conversion rate calculated from the NOx concentration detected by the NOx sensor prior to the instruction time point is lower than a severe reducing agent abnormality threshold.

The first supply valve and the second supply valve may have comparable levels of deterioration over time. Such deterioration includes the case where the NOx conversion rate calculated from the NOx concentration detected by the NOx sensor prior to the instruction time point is equal to or higher than the severe reducing agent abnormality threshold and lower than the supply valve threshold and the case where the NOx conversion rate is lower than the severe reducing agent abnormality threshold. Deterioration that causes the NOx conversion rate to be lower than the severe reducing agent abnormality threshold is called severe supply valve deterioration. Deterioration that causes the NOx conversion rate to be equal to or higher than the severe reducing agent abnormality threshold and lower than the supply valve threshold is called slight deterioration.

Despite that the detected concentration of the reducing agent prior to the instruction time point is normal, when the NOx conversion rate is lower than the severe reducing agent abnormality threshold, it is determined that severe supply valve deterioration occurs. Accordingly, when the NOx conversion rate is lower than the severe reducing agent abnormality threshold, it is determined that sever supply valve deterioration, which is deterioration of both the first supply valve and the second supply valve, occurs.

In the failure determination device for the emission control apparatus of the internal combustion engine according to the above aspect, the controller may determine abnormality of either one of the first supply valve and the second supply valve, when the concentration of the reducing agent detected by the reducing agent concentration sensor is normal, the NOx conversion rate calculated from the NOx concentration detected by the NOx sensor prior to the instruction time point is equal to or higher than the severe reducing agent abnormality threshold, and the NOx conversion rate calculated from the NOx concentration detected by the NOx sensor after elapse of a third specified time duration, which is a shorter time period than the first specified time duration, since the instruction time point is lower than a supply valve threshold. The controller may determine occurrence of slight deterioration that is deterioration of both the first supply valve and the second supply valve and has a lower degree of deterioration than the severe supply valve deterioration, when the NOx conversion rate is equal to or higher than the supply valve threshold.

In the case of slight deterioration, the reducing agent is supplied from the first supply valve and the second supply valve. Giving an instruction to make the supply amount of the reducing agent equal to the criterion supply amount then enables the NOx conversion rate of the entire system to eventually reach or exceed the supply valve threshold.

After elapse of the first specified time duration, even in the case of abnormality of the second supply valve, the NOx conversion rate may become equal to or higher than the supply valve threshold. This makes it difficult to distinguish abnormality of the second supply valve from slight deterioration. The determination is thus based on the NOx conversion rate after elapse of the third specified time duration, which is a shorter time period than the first specified time duration. The third specified time duration is a time duration that causes a difference between the NOx conversion rates in the case of abnormality of the second supply valve and in the case of slight deterioration, since the instruction time point. For example, the third specified time duration may be a time duration that causes the reducing agent to achieve equilibrium in the first NOx catalyst when the first supply valve is normal, since the instruction time point. The third specified time duration may be equal to the second specified time duration described above. In the case of abnormality of the second supply valve, after elapse of the third specified time duration, only a small amount of the reducing agent is supplied to the second NOx catalyst. The NOx conversion rate is thus kept low. In the case of slight deterioration, on the other hand, after elapse of the third specified time duration, a large amount of the reducing agent is supplied to the first NOx catalyst and the second NOx catalyst. The NOx conversion rate is thus increased.

It is determined that slight deterioration occurs, when the NOx conversion rate is equal to or higher than the severe reducing agent abnormality threshold prior to the instruction time point and is equal to or higher than the supply valve threshold after elapse of the third specified time duration since the instruction time point, despite that the detected concentration of the reducing agent is normal.

In the failure determination device for the emission control apparatus of the internal combustion engine according to the above aspect, the controller may determine abnormality of the NOx sensor, when the NOx conversion rate calculated from the NOx concentration detected by the NOx sensor after elapse of a fourth specified time duration since the instruction time point is lower than a sensor threshold, which is a smaller threshold value than a supply valve threshold. The controller may determine abnormality of the first supply valve, when the NOx conversion rate is equal to or higher than the sensor threshold and is lower than the supply valve threshold.

There may be the occurrence of abnormality that provides a higher gain of the NOx sensor than the actual value. In the description below, providing a higher gain of the NOx sensor than the actual value is called gain deviation. In the case of gain deviation of the NOx sensor, the supply amount of the reducing agent has no abnormality. Giving an instruction to make the supply amount of the reducing agent equal to the criterion supply amount accordingly supplies an excess of the reducing agent and thereby causes the reducing agent (ammonia) to flow out of the first NOx catalyst and the second NOx catalyst. This results in decreasing the calculated NOx conversion rate. In the case of abnormality of the first supply valve, on the other hand, no reducing agent is supplied from the first supply valve. This results in increasing the calculated NOx conversion rate. Accordingly, the NOx conversion rate calculated based on the detection of the NOx sensor after elapse of the fourth specified time duration in the case of gain deviation of the NOx sensor is lower than the calculated NOx conversion rate in the case of abnormality of the first supply valve. The fourth specified time duration is a time duration that causes a difference between the NOx conversion rates in the case of abnormality of the first supply valve and in the case of abnormality of the NOx sensor, since the instruction time point. For example, the fourth specified time duration may be a time duration that causes the reducing agent to achieve equilibrium in the second NOx catalyst when the second supply valve is abnormal, since the instruction time point. The fourth specified time duration may be equal to the first specified time duration described above.

A lower limit value of the NOx conversion rate when the NOx sensor has no gain deviation is set to the sensor threshold. When the NOx conversion rate is equal to or higher than the sensor threshold, it is determined that the first supply valve is abnormal. When the NOx conversion rate is lower than the sensor threshold, on the other hand, it is determined that the NOx sensor has gain deviation. The sensor threshold is a smaller value than the severe reducing agent abnormality threshold and the supply valve threshold.

In the failure determination device for the emission control apparatus of the internal combustion engine according to the above aspect, the controller may determine abnormality of the NOx sensor, when the NOx conversion rate calculated from the NOx concentration detected by the NOx sensor prior to the instruction time point is higher than zero and is lower than a severe reducing agent abnormality threshold and the NOx conversion rate calculated from the NOx concentration detected by the NOx sensor after elapse of a fifth specified time duration, which is a shorter time period than the first specified time duration, since the instruction time point is decreased from the NOx conversion rate calculated from the NOx concentration detected by the NOx sensor prior to the instruction time point. The controller may determine occurrence of severe reducing agent abnormality that provides a lower concentration of the reducing agent than the concentration provided by the slight concentration abnormality, when the NOx conversion rate calculated from the NOx concentration detected by the NOx sensor after elapse of the fifth specified time duration is increased from the NOx conversion rate calculated from the NOx concentration detected by the NOx sensor prior to the instruction time point.

When the NOx conversion rate prior to the instruction time point is higher than zero and is lower than the severe reducing agent abnormality threshold, it is expected that neither the first supply valve nor the second supply valve is abnormal but that severe reducing agent abnormality or abnormality of the NOx sensor occurs. In the severe reducing agent abnormality, the concentration of the reducing agent is higher than 0%. The case where the reducing agent is not present is excluded from the severe reducing agent abnormality. The severe reducing agent abnormality threshold is a smaller value than the supply valve threshold as described above and may be a NOx conversion rate in the regular control in the case where either one of the first supply valve and the second supply valve is abnormal. The case where the NOx conversion rate prior to the instruction time point is higher than zero and is lower than the severe reducing agent abnormality threshold includes the case of severe reducing agent abnormality and the case of gain deviation of the NOx sensor.

In the case of gain deviation of the NOx sensor, a large amount of the reducing agent flows out of the second NOx catalyst after elapse of the fifth specified time duration since the instruction time point. Accordingly, in the case of gain deviation of the NOx sensor, the calculated NOx conversion rate is decreased after elapse of the fifth specified time duration since the instruction time point. The fifth specified time duration is a time duration that causes a difference between the NOx conversion rates in the case of severe reducing agent abnormality and in the case of abnormality of the NOx sensor, since the instruction time point. For example, the fifth specified time duration may be a time duration that causes the reducing agent to achieve equilibrium in the first NOx catalyst when the first supply valve is normal, since the instruction time point. The fifth specified time duration may be equal to the second specified time duration described above.

In the case where the NOx conversion rate prior to the instruction time point is higher than zero and severe reducing agent abnormality occurs, on the other hand, the adsorbed amount of the reducing agent is increased after elapse of the specified fifth time duration since the instruction time point. Additionally, the NOx conversion rates of both the first and second NOx catalysts are recovered, so that the calculated NOx conversion rate is increased. Abnormality of the NOx sensor is thus distinguishable from severe reducing agent abnormality, based on a change of the NOx conversion rate prior to the instruction time point and after elapse of the fifth specified time duration since the instruction time point. The second specified time duration, the third specified time duration and the fifth specified time duration are shorter time periods than the first specified time duration and the fourth specified time duration. The second specified time duration, the third specified time duration and the fifth specified time duration may be identical time periods or may be different time periods. The first specified time duration and the fourth specified time duration may be identical time periods or may be different time periods.

When individually controlling a plurality of supply valves is not available, the above aspects of the invention identify which of the supply valves is abnormal with high accuracy, while suppressing cost increase.

### BRIEF DESCRIPTION OF DRAWINGS

FIG. 1 is a diagram illustrating the schematic configuration of an internal combustion engine and its air intake system and its exhaust system according to one embodiment;

FIG. 2 is a graph showing NOx conversion rates in regular control in the case of the normal system, in the case of abnormality of a first supply valve and in the case of abnormality of a second supply valve;

FIG. 3 is a graph showing NOx conversion rates in increase control in the case of abnormality of the first supply valve and in the case of abnormality of the second supply valve, after elapse of a time duration that causes the reducing agent to achieve equilibrium in the second NOx catalyst when the second supply valve is abnormal, since an instruction time point;

FIG. 4 is a time chart showing one example of variations of adsorbed amounts of the reducing agent to the respective catalysts and a variation in NOx conversion rate in the case of abnormality of the second supply valve in the increase control;

FIG. 5 is a time chart showing one example of variations of the adsorbed amounts of the reducing agent to the respective catalysts and a variation in NOx conversion rate in the case of abnormality of the first supply valve in the increase control;

FIG. 6 is a flowchart showing a flow of abnormality determination according to Embodiment 1;

FIG. 7 is a flowchart showing a supply valve determination process;

FIG. 8 is a diagram showing types of abnormalities as objects of determination according to Embodiment 2;

FIG. 9 is a graph showing the relationship between the NOx conversion rate and a severe reducing agent abnormality threshold in the regular control, in the case of the normal system, in the case of slight abnormality and in the case of severe reducing agent abnormality;

FIG. 10 is a graph showing NOx concentrations in the regular control in the case of abnormality of the first supply valve, in the case of abnormality of the second supply valve and in the case of slight concentration abnormality;

FIG. 11 is a graph showing NOx conversion rates in the increase control in the case of abnormality of the first supply valve, in the case of abnormality of the second supply valve and in the case of slight concentration abnormality after elapse of a time duration that causes the reducing agent to achieve equilibrium in the first NOx catalyst when the first supply valve is normal, since the instruction time point;

FIG. 12 is a time chart showing one example of variations of the adsorbed amounts of the reducing agent to the respective catalysts and a variation in NOx conversion rate in the case of slight concentration abnormality in the increase control;

FIG. 13 is a flowchart showing a flow of abnormality determination according to Embodiment 2;

FIG. 14 is a flowchart showing a slight abnormality determination process;

FIG. 15 is a flowchart showing a severe reducing agent abnormality determination process;

FIG. 16 is a flowchart showing a flow of abnormality determination according to Embodiment 3;

FIG. 17 is a flowchart showing a slight abnormality determination process performed at step S604 in FIG. 16;

FIG. 18 is a flowchart showing a concentration abnormality determination process performed at step S602 in FIG. 16;

FIG. 19 is a graph showing the relationship between the NOx conversion rate and the severe reducing agent abnormality threshold in the regular control, in the case of abnormality of the first supply valve and in the case of gain deviation of the second NOx sensor;

FIG. 20 is a graph showing the relationship between the NOx conversion rate and the severe reducing agent abnormality threshold in the regular control, in the case of severe concentration abnormality and in the case of gain deviation of the second NOx sensor;

FIG. 21 is a graph showing the relationship between the NOx conversion rate and the severe reducing agent abnormality threshold after elapse of a supply valve abnormality determination time duration since the instruction time point in the increase control, in the case of abnormality of the first supply valve and in the case of gain deviation of the second NOx sensor;

FIG. 22 is a graph showing the relationship between the NOx conversion rate and the severe reducing agent abnormality threshold after elapse of a slight concentration abnormality determination time duration since the instruction time point in the increase control, in the case of severe concentration abnormality and in the case of gain deviation of the second NOx sensor;

FIG. 23 is a flowchart showing a flow of abnormality determination according to Embodiment 4;

FIG. 24 is a flowchart showing a slight abnormality determination process performed at step S901 in FIG. 23; and

FIG. 25 is a flowchart showing a severe reducing agent abnormality determination process performed at step S902 in FIG. 23.

### DESCRIPTION OF EMBODIMENTS

The following illustratively describes some aspects of the invention in detail based on embodiments with reference to the drawings. The dimensions, the materials, the shapes, the positional relationships and the like of the respective components described in the following embodiments are only for the purpose of illustration and not intended at all to limit the scope of the invention to such specific descriptions.

### Embodiment 1

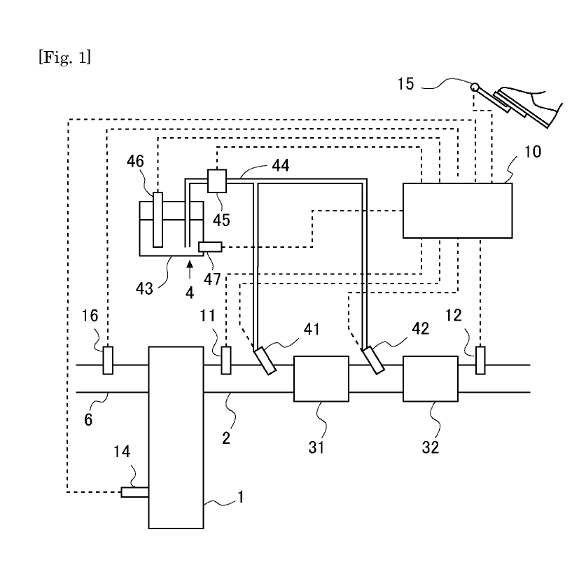

FIG. 1 is a diagram illustrating the schematic configuration of an internal combustion engine 1 and its air intake system and its exhaust system according to one embodiment. The internal combustion engine 1 is a diesel engine for vehicle driving. The internal combustion engine 1 is connected with an exhaust conduit 2. The exhaust conduit 2 is provided with a first supply valve 41, a first NOx catalyst 31, a second supply valve 42 and a second NOx catalyst 32 sequentially from an upstream side to a downstream side along a flow direction of exhaust emission. The first NOx catalyst 31 and the second NOx catalyst 32 are selective reduction NOx catalysts that use ammonia as a reducing agent to selectively reduce NOx in the exhaust emission. The first NOx catalyst 31 of this embodiment corresponds to the first selective reduction NOx catalyst of the invention. The second NOx catalyst 32 of this embodiment corresponds to the second selective reduction NOx catalyst of the invention.

The first supply valve 41 and the second supply valve 42 form part of a reducing agent supply device 4. The reducing agent supply device 4 also includes an urea tank 43, a reducing agent supply path 44, a pump 45, a reducing agent quantity sensor 46 and a reducing agent concentration sensor 47, in addition to the first supply valve 41 and the second supply valve 42.

The urea tank 43 stores urea water. The urea water is hydrolyzed to ammonia by heat of the exhaust emission or heat from the first NOx catalyst 31 or the second NOx catalyst 32 and is adsorbed to the first NOx catalyst 31 or the second NOx catalyst 32. This ammonia is used as the reducing agent in the first NOx catalyst 31 or the second NOx catalyst 32. The reducing agent supply path 44 has one end that is connected with the urea tank 43 and the other end that forks to two respectively connected with the first supply valve 41 and the second supply valve 42. The reducing agent supply path 44 is arranged to supply urea water to the first supply valve 41 and the second supply valve 42. The pump 45 is located in the middle of the reducing agent supply path 44 on the urea tank 43-side of the branching position of the reducing agent supply path 44 to pump out urea water toward the first supply valve 41 and the second supply valve 42. The reducing agent quantity sensor 46 serves to detect the amount of urea water stored in the urea tank 43. The reducing agent concentration sensor 47 serves to detect the concentration of urea water stored in the urea tank 43. The configuration of this embodiment may not necessarily include the reducing agent concentration sensor 47. In the description of the embodiment, ammonia and urea water may be called reducing agent.

Additionally, a first NOx sensor 11 is provided upstream of the first supply valve 41 to detect NOx in the exhaust emission flowing into the first NOx catalyst 31. A second NOx sensor 12 is provided downstream of the second NOx catalyst 32 to detect NOx in the exhaust emission flowing out of the second NOx catalyst 32. The first NOx sensor 11 and the second NOx sensor 12 detect ammonia as well as NOx.

The internal combustion engine 1 is also connected with an air intake conduit 6. An air flowmeter 16 is located in the middle of the air intake conduit 6 to detect the amount of intake air taken into the internal combustion engine 1.

The internal combustion engine 1 is further provided with an ECU 10 as an electronic control unit. The ECU 10 serves to control, for example, the operating conditions of the internal combustion engine 1 and an emission control apparatus. The ECU 10 is electrically connected with a crank position sensor 14 and an accelerator position sensor 15, in addition to the first NOx sensor 11, the second NOx sensor 12 and the air flowmeter 16 described above to receive output valves from the respective sensors. The ECU 10 of this embodiment corresponds to the controller of the invention.

The ECU 10 obtains the operating conditions of the internal combustion engine, for example, an engine rotation speed based on the detection result of the crank position sensor 14 and an engine load based on the detection result of the accelerator position sensor 15. According to this embodiment, the NOx concentration in the exhaust emission flowing into the first NOx catalyst 31 is detectable by the first NOx sensor 11. The NOx concentration in the exhaust emission discharged from the internal combustion engine 1 (i.e., exhaust emission prior to catalytic conversion by the first NOx catalyst 31, in other words, exhaust emission flowing into the first NOx catalyst 31) is related to the operating conditions of the internal combustion engine 1 and may thus be estimated based on the operating conditions of the internal combustion engine 1 described above.

The ECU 10 sends an identical signal to the first supply valve 41 and the second supply valve 42 to control the first supply valve 41 and the second supply valve 42. More specifically, the ECU 10 gives an identical instruction with regard to valve opening and closing to the first supply valve 41 and the second supply valve 42. Accordingly, the first supply valve 41 and the second supply valve 42 supply the reducing agent at an identical timing. The ECU 10 performs regular control to supply the reducing agent from the first supply valve 41 and the second supply valve 42 with regard to the amount of NOx flowing into the first NOx catalyst 31 such that the NOx conversion rate of the entire system is within a target range. Accordingly the reducing agent is supplied from the first supply valve 41 and the second supply valve 42 according to the amount of NOx discharged from the internal combustion engine 1. The regular control estimates the adsorbed amount of the reducing agent in each of the first NOx catalyst 31 and the second NOx catalyst 32. The regular control may supply the reducing agent from the first supply valve 41 and the second supply valve 42 such that the respective NOx catalysts 31 and 32 have fixed adsorbed amounts of the reducing agent. The adsorbed amount of the reducing agent by the first NOx catalyst 31 is estimated using a model, based on the amount of the reducing agent supplied from the first supply valve 41, the NOx conversion rate of the first NOx catalyst 31 and the amount of the reducing agent flowing out of the first NOx catalyst 31. The adsorbed amount of the reducing agent by the second NOx catalyst 32 is estimated using a model, based on the amount of the reducing agent supplied from the second supply valve 42, the NOx conversion rate of the second NOx catalyst 32, the amount of the reducing agent flowing out of the second NOx catalyst 32 and the amount of the reducing agent flowing out of the first NOx catalyst 31. The NOx conversion rate of the first NOx catalyst 31, the NOx conversion rate of the second NOx catalyst 32, the amount of the reducing agent flowing out of the first NOx catalyst 31 and the amount of the reducing agent flowing out of the second NOx catalyst 32 are estimated based on the temperature and another factor. In this estimation, it is assumed that both the first supply valve 41 and the second supply valve 42 are normal.

The ECU 10 determines whether the first supply valve 41 is abnormal or the second supply valve 42 is abnormal. This embodiment may be configured to confirm that the other devices, components and the like have no abnormality by any known means. For example, it may be configured that neither the first NOx catalyst 31 nor the second NOx catalyst 32 is abnormal.

The ECU 10 computes the NOx conversion rate of the entire system of the first NOx catalyst 31 and the second NOx catalyst 32, based on the NOx concentration detected by the first NOx sensor 11 (or NOx concentration estimated from the operating conditions of the internal combustion engine 1) and the NOx concentration detected by the second NOx sensor 12. The NOx concentration detected by the first NOx sensor 11 denotes the NOx concentration in the exhaust emission flowing into the first NOx catalyst 31 (upstream-side NOx concentration). The NOx concentration detected by the second NOx sensor 12 denotes the NOx concentration in the exhaust emission flowing out of the second NOx catalyst 32 (downstream-side NOx concentration). In the description below, unless otherwise specified, the NOx conversion rate means the NOx conversion rate of the entire system. The NOx conversion rate indicates a ratio of the NOx concentration decreased by conversion of NOx in the first NOx catalyst 31 and the second NOx catalyst 32 to the NOx concentration in the exhaust emission flowing into the first NOx catalyst 31 and is calculated by the following equation:

NOx conversion rate=(upstream-side NOx concentration−downstream-side NOx concentration)/upstream-side NOx concentration

In the equation, the upstream-side NOx concentration denotes the NOx concentration in the exhaust emission flowing into the first NOx catalyst 31, and the downstream-side NOx concentration denotes the NOx concentration in the exhaust emission flowing out of the second NOx catalyst 32.

When the NOx conversion rate is lower than a lower limit in a normal range (also called normal threshold), it is expected that one of the first supply valve 41 and the second supply valve 42 is abnormal. It is, however, difficult to identify which of abnormality of the first supply valve 41 and abnormality of the second supply valve 42. In the regular control that supplies the reducing agent from the first supply valve 41 and the second supply valve 42 according to the amount of NOx discharged from the internal combustion engine 1, the case where the first supply valve 41 is abnormal and the case where the second supply valve 42 is abnormal may have substantially the same NOx conversion rates. This embodiment is accordingly configured to identify which of abnormality of the first supply valve 41 and abnormality of the second supply valve 42, on the assumption that the first supply valve 41 and the second supply valve 42 do not become abnormal simultaneously. The abnormality of the first supply valve 41 or the second supply valve 42 includes no supply of the reducing agent and only little supply of the reducing agent. The normal threshold of this embodiment corresponds to the supply valve threshold of the invention.

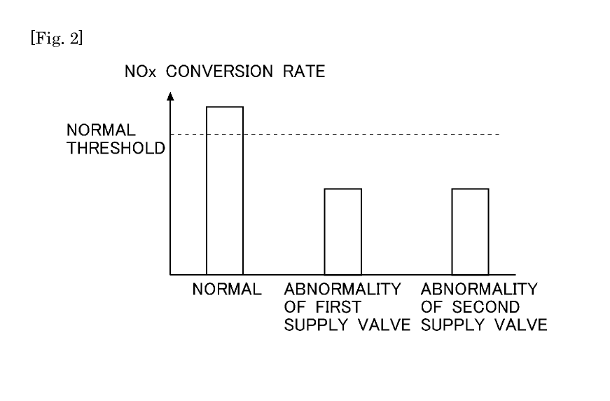

FIG. 2 is a graph showing NOx conversion rates in the regular control, in the case of the normal system, in the case of abnormality of the first supply valve 41 and in the case of abnormality of the second supply valve 42. The normal threshold in FIG. 2 denotes a lower limit value of the NOx conversion rate in the case of the normal system. FIG. 2 shows the NOx conversion rates after elapse of a sufficient time duration since a start of supplying the reducing agent and when the NOx conversion rate is supposed to be equal to or higher than the normal threshold in the normal state of the system. The case of abnormality of the first supply valve 41 and the case of abnormality of the second supply valve 42 may have substantially the same NOx conversion rates as shown in FIG. 2. It is difficult to identify which of abnormality of the first supply valve 41 and abnormality of the second supply valve 42, on the basis of such NOx conversion rates

This embodiment, on the other hand, notes a variation in NOx conversion rate after the supply amount of the reducing agent is increased to a criterion supply amount. The ECU 10 increases the instruction value of the supply amount of the reducing agent. In the case where the first supply valve 41 or the second supply valve 42 is abnormal, however, the actual supply amount of the reducing agent from the abnormal supply valve is not increased. The criterion supply amount is a larger supply amount of the reducing agent than the amount in the regular control. In other words, the criterion supply amount is a larger supply amount of the reducing agent than the amount of the reducing agent required for conversion of NOx. The criterion supply amount is accordingly a supply amount of the reducing agent that causes the reducing agent to flow out of the first NOx catalyst 31 and the second NOx catalyst 32 in the case where both the first supply valve 41 and the second supply valve 42 are normal. A time point when the ECU 10 gives an instruction to the first supply valve 41 and the second supply valve 42 to make the supply amount of the reducing agent equal to the criterion supply amount is called “instruction time point” in the description below. Control performed by the ECU 10 to give an instruction to the first supply valve 41 and the second supply valve 42 to make the supply amount of the reducing agent equal to the criterion supply amount is called “increase control” in the description below.

### <Determination of Abnormality of First Supply Valve 41 and Second Supply Valve 42>

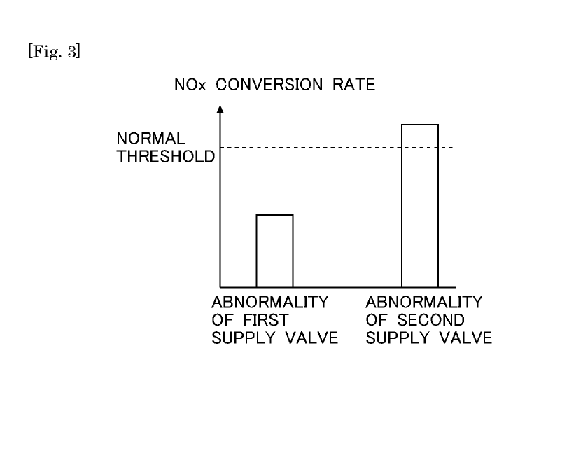

FIG. 3 is a graph showing NOx conversion rates in the increase control in the case of abnormality of the first supply valve 41 and in the case of abnormality of the second supply valve 42, after elapse of a time duration that causes the reducing agent to achieve equilibrium in the second NOx catalyst 32 when the second supply valve 42 is abnormal, since the instruction time point.

In the case of abnormality of the first supply valve 41, the first supply valve 41 fails to supply the reducing agent to the first NOx catalyst 31, so that the first NOx catalyst 31 fails to convert NOx. The second supply valve 42 is, however, normal, so that the second NOx catalyst 32 works to convert NOx. In this state, an excess of the reducing agent is supplied to the second NOx catalyst 32. The reducing agent accordingly flows out of the second NOx catalyst 32 even before the reducing agent achieves equilibrium in the second NOx catalyst 32. The second NOx sensor 12 detects ammonia other than NOx as described above. The presence of ammonia in the exhaust emission increases the detection value of the second NOx sensor 12. This results in decreasing the NOx conversion rate calculated based on the detection value of the second NOx sensor 12. Accordingly, in the case of abnormality of the first supply valve 41, the NOx conversion rate of the entire system is relatively low.

In the case of abnormality of the second supply valve 42, on the other hand, the second supply valve 42 fails to supply the reducing agent to the second NOx catalyst 32. The first supply valve 41, however, supplies the reducing agent to the first NOx catalyst 31, so that the first NOx catalyst 31 works to convert NOx. In this state, the amount of the reducing agent supplied from the first supply valve 41 is larger than the amount in the regular control or more specifically than the amount of the reducing agent required for conversion of NOx. Part of the reducing agent supplied from the first supply valve 41 accordingly flows out of the first NOx catalyst 31. The flow-out reducing agent is supplied to the second NOx catalyst 32, so that the reducing agent achieves equilibrium in the second NOx catalyst 32 after elapse of a certain time duration. FIG. 3 shows the NOx conversion rates after elapse of the time duration that causes the reducing agent to achieve equilibrium in the second NOx catalyst 32.

In the case where the second supply valve 42 is abnormal, the second supply valve 42 fails to supply the reducing agent to the second NOx catalyst 32. The reducing agent supplied from the first supply valve 41 is, however, adsorbed to the second NOx catalyst 32, so that the second NOx catalyst 32 works to convert NOx. In this state, both the first NOx catalyst 31 and the second NOx catalyst 32 work to convert NOx, so that the NOx conversion rate of the entire system may become equal to or higher than the normal threshold.

In the increase control, after elapse of a time duration that causes a distinguishable difference between the NOx conversion rate in the case of abnormality of the first supply valve 41 and the NOx conversion rate in the case of abnormality of the second supply valve 42, since the instruction time point, it is determinable which of the first supply valve 41 and the second supply valve 42 is abnormal, based on the NOx conversion rate at this moment. The time duration that causes a distinguishable difference between the NOx conversion rate in the case of abnormality of the first supply valve 41 and the NOx conversion rate in the case of abnormality of the second supply valve 42 may be set to a time duration that causes the reducing agent to achieve equilibrium in the second NOx catalyst 32 when the second supply valve 42 is abnormal (hereinafter referred to as “supply valve abnormality determination time duration”). The NOx conversion rate is equal to or higher than the normal threshold in the case of abnormality of the second supply valve 42, while being lower than the normal threshold in the case of abnormality of the first supply valve 41. According to this embodiment, when the NOx conversion rate after elapse of the supply valve abnormality determination time duration since the instruction time point in the increase control is equal to or higher than the normal threshold, it is determined that the second supply valve 42 is abnormal and the first supply valve 41 is normal. When the NOx conversion rate after elapse of the supply valve abnormality determination time duration since the instruction time point in the increase control is lower than the normal threshold, on the other hand, it is determined that the second supply valve 42 is normal and the first supply valve 41 is abnormal. The supply valve abnormality determination time duration is related to the NOx conversion rate in the regular control, the criterion supply amount, the amount of the reducing agent adsorbable to the first NOx catalyst 31 and the amount of the reducing agent adsorbable to the second NOx catalyst 32. These relationships may be determined in advance by experiment, by simulation or the like. The supply valve abnormality determination time duration of this embodiment corresponds to the first specified time duration of the invention.

### <Time Chart in Determination of Abnormality>

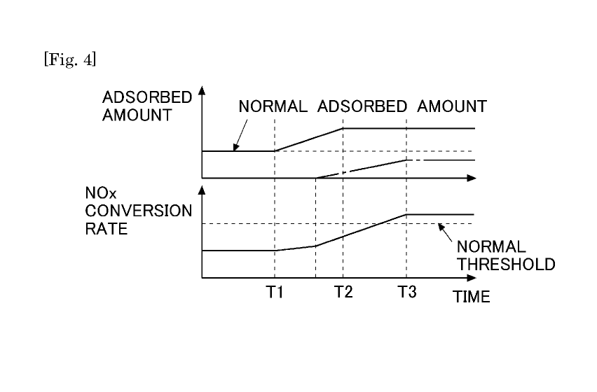

FIG. 4 is a time chart showing one example of variations of the adsorbed amounts of the reducing agent to the respective catalysts and a variation in NOx conversion rate in the case of abnormality of the second supply valve 42 in the increase control. T1 represents the instruction time point in the increase control. T2 represents a time point after elapse of a time duration that causes the reducing agent to achieve equilibrium in the first NOx catalyst 31 since T1 in the case where the first supply valve 41 is normal. T3 represents a time point after elapse of the supply valve abnormality determination time duration since T1. In other words, T2 also represents a time point after elapse of a time duration that causes the reducing agent to achieve equilibrium in the second NOx catalyst 32 since T1 in the case where the second supply valve 42 is normal. The time duration between T1 and T2 is shorter than the time duration between T1 and T3. In the adsorbed amount chart, a solid-line curve shows the adsorbed amount of the reducing agent to the first NOx catalyst 31, and a one dot-chain line curve shows the adsorbed amount of the reducing agent to the second NOx catalyst 32. A normal adsorbed amount denotes a lower limit value of the adsorbed amount of the reducing agent to each of the first and the second NOx catalysts 31 and 32 in the case where both the first NOx catalyst 31 and the second NOx catalyst 32 are normal.

In the case where the second supply valve 42 is abnormal, an adequate amount of the reducing agent is supplied from the first supply valve 41 to the first NOx catalyst 31 even before T1, so that the adsorbed amount of the reducing agent to the first NOx catalyst 31 is close to the normal adsorbed amount. Substantially no reducing agent is, however, supplied to the second NOx catalyst 32 before T1, so that the adsorbed amount of the reducing agent to the second NOx catalyst 32 before T1 is approximately zero. In the case where the second supply valve 42 is abnormal, the amount of the reducing agent adsorbed to the second NOx catalyst 32 does not increase immediately with an increase in supply amount of the reducing agent at the time point T1. More specifically, when the reducing agent flows out of the first NOx catalyst 31 after a certain increase in amount of the reducing agent adsorbed to the first NOx catalyst 31, the amount of the reducing agent adsorbed to the second NOx catalyst 32 starts increasing. The NOx conversion rate increases with an increase in amount of the reducing agent adsorbed to the first NOx catalyst 31. At the time point T2, however, only an insufficient amount of the reducing agent is adsorbed to the second NOx catalyst 32, so that the NOx conversion rate is lower than the normal threshold. At the time point T3 after elapse of the supply valve abnormality determination time duration, the reducing agent achieves equilibrium in the second NOx catalyst 32. In this state, the amount of the reducing agent adsorbed to the second NOx catalyst 32 is less than the normal adsorbed amount. At the time point T3, however, an excess amount of the reducing agent is adsorbed to the first NOx catalyst 31, so that the NOx conversion rate of the first NOx catalyst 31 is increased. Accordingly, even when the second NOx catalyst 32 has a slightly low NOx conversion rate, the NOx conversion rate at the time point T3 becomes equal to or higher than the normal threshold.

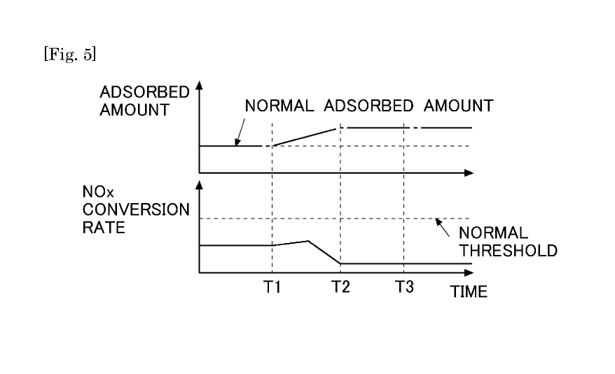

FIG. 5 is a time chart showing one example of variations of the adsorbed amounts of the reducing agent to the respective catalysts and a variation in NOx conversion rate in the case where the first supply valve 41 is abnormal in the increase control. The time points T1, T2 and T3 in FIG. 5 are identical with the time point T1, T2 and T3 in FIG. 4.

In the case where the first supply valve 41 is abnormal, the increase control does not increase the amount of the reducing agent adsorbed to the first NOx catalyst 31. Accordingly, the amount of the reducing agent adsorbed to the first NOx catalyst 31 is consistently equal to zero. An adequate amount of the reducing agent is, however, supplied to the second NOx catalyst 32 even before T1, so that the adsorbed amount of the reducing agent to the second NOx catalyst 32 is close to the normal adsorbed amount. The amount of the reducing agent adsorbed to the second NOx catalyst 32 starts increasing at the instruction time point T1 in the increase control. The NOx conversion rate has a temporary increase with an increase in amount of the reducing agent adsorbed to the second NOx catalyst 32. Before the time point T2, however, the reducing agent flows out of the second NOx catalyst 32, and the second NOx sensor 12 detects ammonia. This results in decreasing the NOx conversion rate that is calculated based on the NOx concentration detected by the second NOx sensor 12. At the time point T2, the reducing agent achieves equilibrium in the second NOx catalyst 32. After the time point T2, ammonia continues flowing out of the second NOx catalyst 32, and the NOx conversion rate is kept low. Accordingly, in the case where the first supply valve 41 is abnormal, the NOx conversion rate is kept lower than the normal threshold over the entire time period.

In the increase control, when the NOx conversion rate is equal to or higher than the normal threshold at the time point T3, it is determined that the second supply valve 42 is abnormal. When the NOx conversion rate is lower than the normal threshold at the time point T3, on the other hand, it is determined that the first supply valve 41 is abnormal. According to one modification, it may be determined that the second supply valve 42 is abnormal at a time point when the NOx conversion rate becomes equal to or higher than the normal threshold.

### <Flow of Abnormality Determination>

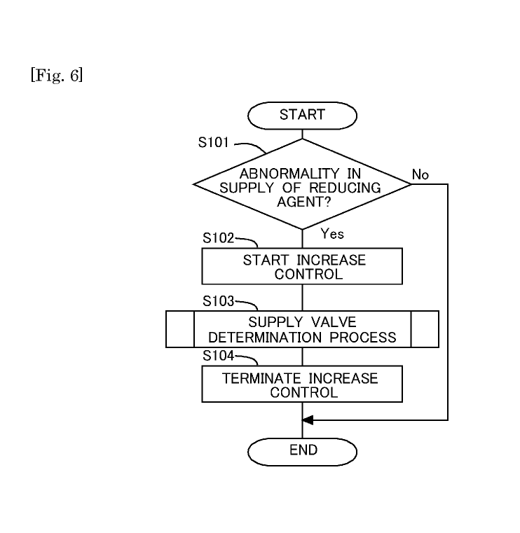

FIG. 6 is a flowchart showing a flow of abnormality determination according to this embodiment. This flow is performed at predetermined time intervals by the ECU 10.

At step S101, the ECU 10 determines whether abnormality occurs in supply of the reducing agent. More specifically, according to this embodiment, the ECU 10 determines whether either the first supply valve 41 or the second supply valve 42 is abnormal at step S101. The abnormality in supply of the reducing agent results in decreasing the NOx conversion rate of the entire system. When the NOx conversion rate is lower than the normal threshold, it is determined that abnormality occurs in supply of the reducing agent. The normal threshold is determined in advance by experiment, by simulation or the like, as the lower limit value of the NOx conversion rate in the normal state of the system. The supply amount of the reducing agent in this state is a supply amount of the reducing agent in the regular control prior to the instruction time point in the increase control.

It may be confirmed in advance that the other devices, components and the like including the first NOx catalyst 31 and the second NOx catalyst 32 have no abnormality. Such confirmation may be performed at step S101 or prior to execution of this flow. For example, in the normal state, the first NOx catalyst 31 and the second NOx catalyst 32 generate heat by absorption of water. It may be determined that the first NOx catalyst 31 and the second NOx catalyst 32 are normal, based on temperature increases of the first NOx catalyst 31 and the second NOx catalyst 32 in the case where water is likely to flow into the first NOx catalyst 31 or the second NOx catalyst 32, for example, at a start of the internal combustion engine 1. In the case where the first NOx catalyst 31 or the second NOx catalyst 32 is abnormal, the adsorption power of the reducing agent in the first NOx catalyst 31 or the second NOx catalyst 32 is decreased. In the event of a failure in adsorption of the reducing agent despite that the normal first NOx catalyst 31 and second NOx catalyst 32 are capable of adsorbing the reducing agent, it may be determined that either the first NOx catalyst 31 or the second NOx catalyst 32 is abnormal. It may be confirmed that no abnormality occurs in any device, component or the like other than the reducing agent supply device 4 and the reducing agent, by any other known technique. In the case of an affirmative answer at step S101, the flow goes to step S102. In the case of a negative answer at step S101, on the other hand, the flow is terminated.

At step S102, the ECU 10 starts the increase control or more specifically gives an instruction to make the supply amount of the reducing agent equal to the criterion supply amount. The increase control increases the supply amount of the reducing agent such as to make the reducing agent achieve equilibrium in the first NOx catalyst 31 and in the second NOx catalyst 32 and make the reducing agent flow out of the first NOx catalyst 31 and the second NOx catalyst 32 in the case where both the first supply valve 41 and the second supply valve 42 are normal. The supply amount of the reducing agent is increased by increasing the supply time of the reducing agent. The increase control increases the instruction value of the supply amount of the reducing agent given by the ECU 10. The same instruction is given simultaneously to the first supply valve 41 and the second supply valve 42. Since either the first supply valve 41 or the second supply valve 42 is abnormal in this case, the actual supply amount of the reducing agent is not increased but is kept zero in the abnormal supply valve irrespective of the increase in instruction value of the supply amount of the reducing agent. The flow goes to step S103 after conclusion of step S102.

At step S103, the ECU 10 performs a supply valve determination process or more specifically determines whether the first supply valve 41 is abnormal or the second supply valve 42 is abnormal. This supply valve determination process will be described below in detail. The flow goes to step S104 after conclusion of step S103.

At step S104, the ECU 10 terminates the increase control or more specifically returns the supply amount of the reducing agent from the criterion supply amount to the supply amount in the regular control. This starts supplying the reducing agent according to the amount of NOx discharged from the internal combustion engine 1. The flow is terminated after conclusion of step S104.

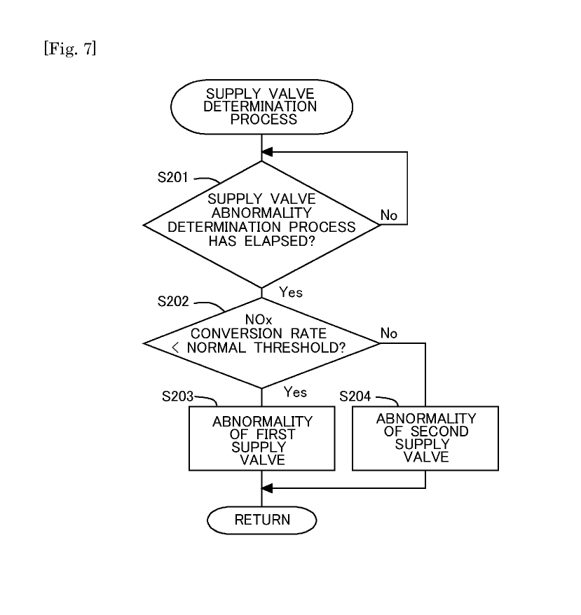

The following describes the supply valve determination process performed at step S103. FIG. 7 is a flowchart showing the supply valve determination process.

At step S201, the ECU 10 determines whether the supply valve abnormality determination time duration has elapsed since the time point when the increase control is started at step S102 (instruction time point). More specifically, the ECU 10 determines whether a time duration that allows for identification of which of abnormality of the first supply valve 41 and abnormality of the second supply valve 42 at step S201. In the case of an affirmative answer at step S201, the flow goes to step S202. In the case of a negative answer at step S201, on the other hand, the flow repeats step S201.

At step S202, the ECU 10 determines whether the NOx conversion rate at the current moment is lower than the normal threshold. More specifically, the ECU 10 determines whether the NOx conversion rate at the time point after elapse of the supply valve abnormality determination time duration since the instruction time point in the increase control (T3 in FIG. 4 or FIG. 5) is lower than the normal threshold. After elapse of the supply valve abnormality determination time duration, the NOx conversion rate becomes equal to or higher than the normal threshold in the case of abnormality of the second supply valve 42, while being lower than the normal threshold in the case of abnormality of the first supply valve 41. The determination result of this step accordingly identifies which of abnormality of the first supply valve 41 and abnormality of the second supply valve 42. In the case of an affirmative answer at step S202, the flow goes to step S203 to determine that the first supply valve 41 is abnormal. In the case of a negative answer at step S202, on the other hand, the flow goes to step S204 to determine that the second supply valve 42 is abnormal. Subsequently, this flow is terminated, and the supply valve determination process of step S103 is terminated.

As described above, when abnormality occurs in supply of the reducing agent, this embodiment identifies which of abnormality of the first supply valve 41 and abnormality of the second supply have 42.

### Embodiment 2

This embodiment is configured to identify which of abnormality of the first supply valve 41, abnormality of the second supply valve 42 and abnormality of the reducing agent when abnormality occurs in the system. Embodiment 1 performs the abnormality determination on the assumption that no abnormality occurs in any device, component or the like other than the first supply valve 41 and the second supply valve 42. This embodiment additionally determines occurrence of abnormality in the reducing agent. According to this embodiment, it may be confirmed that no abnormality occurs in any other device, component or the like, by any known means. For example, it may be confirmed that neither the first NOx catalyst 31 nor the second NOx catalyst 32 is abnormal. The configuration of the other devices, components and the like is identical with that of Embodiment 1 and is not specifically described here.

In the case where the reducing agent is abnormal, the NOx conversion rate in the regular control may be decreased, as in the case where the first supply valve 41 or the second supply valve 42 is abnormal. When the NOx conversion rate is lower than the normal threshold, it is expected that one of the reducing agent, the first supply valve 41 and the second supply valve 42 is abnormal according to this embodiment. It is, however, difficult to identify which of abnormality of the reducing agent, abnormality of the first supply valve 41 and abnormality of the second supply valve 42. The abnormality of the reducing agent is divided into slight concentration abnormality in which the NOx conversion rate in the regular control is equal to or higher than a severe reducing agent abnormality threshold and severe concentration abnormality in which the NOx conversion rate is lower than the severe reducing agent abnormality threshold.

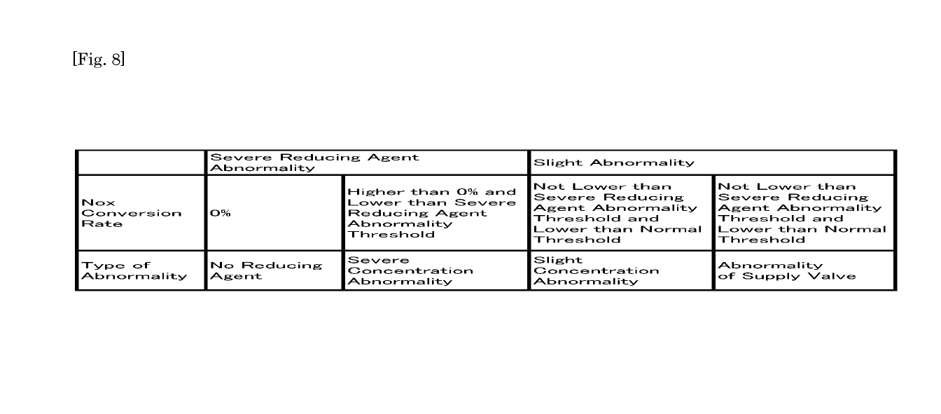

FIG. 8 is a diagram showing types of abnormalities as objects of determination according to this embodiment. The NOx conversion rate in FIG. 8 shows the value in the regular control. Severe reducing agent abnormality includes the case of no reducing agent and the case of the concentration of the reducing agent that provides the NOx conversion rate in the regular control that is higher than 0% and is lower than the severe reducing agent abnormality threshold (hereinafter referred to as “severe concentration abnormality”). The case of no reducing agent may be the case where no reducing agent (urea water) is stored in the urea tank 43 (i.e., the reducing agent is used up and the remaining quantity of the reducing agent is zero) or may be the case where the concentration of the reducing agent (urea water) is 0% (i.e., in the case of storage of water or another liquid).

The slight concentration abnormality indicates the case where the concentration of the reducing agent is higher than that in the severe concentration abnormality but does not reach a specified concentration. The slight concentration abnormality, abnormality of the first supply valve 41 and abnormality of the second supply valve 42 may be called slight abnormality in the description below. The concentration of the reducing agent in the case of slight concentration abnormality provides the NOx conversion rate that is lower than the normal threshold in the regular control but may become equal to or higher than the normal threshold in the increase control. Accordingly, the severe reducing agent abnormality threshold denotes a NOx conversion rate in the regular control at a lower limit value of the concentration of the reducing agent that is likely to increase the NOx conversion rate to the normal threshold in the increase control. In other words, the severe reducing agent abnormality threshold denotes a NOx conversion rate in the regular control in the case where the reducing agent has no abnormality and either the first supply valve 41 or the second supply valve 42 is abnormal.

When any of such abnormalities occurs, the NOx conversion rate is decreased in the regular control in any case. When the NOx conversion rate becomes lower than the normal threshold in the regular control, this embodiment identifies the abnormality on the assumption that any two or more of the reducing agent, the first supply valve 41 and the second supply valve 42 do not become abnormal simultaneously.

### <Determination of Severe Reducing Agent Abnormality>

In the case of severe reducing agent abnormality (in the case of no reducing agent and in the case of severe concentration abnormality), the NOx conversion rate in the regular control is lower than the severe reducing agent abnormality threshold. In the case of slight concentration abnormality, the reducing agent is supplied to some extent, although the supply amount of the reducing agent is small. The NOx conversion rate in the regular control is accordingly equal to or higher than the severe reducing agent abnormality threshold. In the case where either the first supply valve 41 or the second supply valve 42 is abnormal, the NOx conversion rate in the regular control is also equal to or higher than the severe reducing agent abnormality threshold.

Accordingly, the severe reducing agent abnormality is detected when the NOx conversion rate in the regular control is lower than the severe reducing agent abnormality threshold. The severe reducing agent abnormality threshold may be determined in advance by experiment, by simulation or the like.

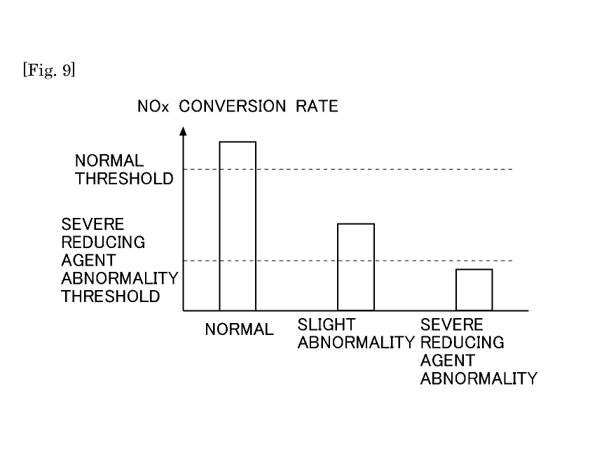

FIG. 9 is a graph showing the relationship between the NOx conversion rate and the severe reducing agent abnormality threshold in the regular control, in the case of the normal system, in the case of slight abnormality and in the case of severe reducing agent abnormality. In the case of the normal system, the NOx conversion rate is equal to or higher than the normal threshold. In the case of slight abnormality, the NOx conversion rate is equal to or higher than the severe reducing agent abnormality threshold and is lower than the normal threshold. In the case of severe reducing agent abnormality, the NOx conversion rate is lower than the severe reducing agent abnormality threshold. The severe reducing agent abnormality and the slight abnormality are distinguishable from each other by comparison between the NOx conversion rate and the severe reducing agent abnormality threshold.

In the case of severe reducing agent abnormality, when the NOx conversion rate is higher than 0%, at least some reducing agent is supplied. The concentration of the reducing agent is accordingly not 0% but is abnormally low to provide the NOx conversion rate that is lower than the severe reducing agent abnormality threshold (i.e., severe concentration abnormality). In the case of severe reducing agent abnormality, when the NOx conversion rate is 0%, it is expected that no reducing agent is present. When the NOx conversion rate is 0% and the amount of the reducing agent detected by the reducing agent quantity sensor 46 is equal to zero, it is expected that the reducing agent stored in the urea tank 43 is used up. When the NOx conversion rate is 0% and the amount of the reducing agent detected by the reducing agent quantity sensor 46 is not equal to zero, it is expected that the concentration of the reducing agent is 0%, in other words, that liquid other than the reducing agent (for example, water) is stored in the urea tank 43.

### <Determination of Slight Concentration Abnormality>

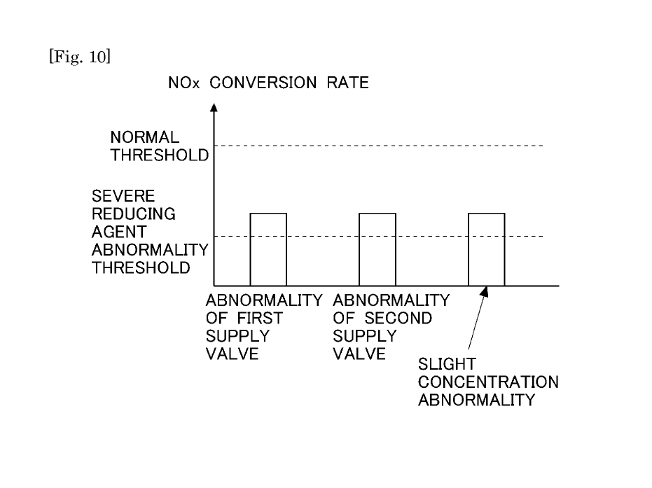

The following describes the case where abnormality but severe reducing agent abnormality occurs in the system, i.e., the case of slight abnormality. This indicates one of slight concentration abnormality, abnormality of the first supply valve 41 or abnormality of the second supply valve 42. FIG. 10 is a graph showing NOx concentrations in the regular control in the case of abnormality of the first supply valve 41, in the case of abnormality of the second supply valve 42 and in the case of slight concentration abnormality. FIG. 10 shows the NOx conversion rates after elapse of a sufficient time duration since a start of supplying the reducing agent and when the NOx conversion rate is supposed to be equal to or higher than the normal threshold in the normal state of the system. The case of slight concentration abnormality, the case of abnormality of the first supply valve 41 and the case of abnormality of the second supply valve 42 may have substantially the same NOx conversion rates as shown in FIG. 10. It is difficult to identify which of abnormality of the reducing agent, abnormality of the first supply valve 41 and abnormality of the second supply valve 42, on the basis of such NOx conversion rates

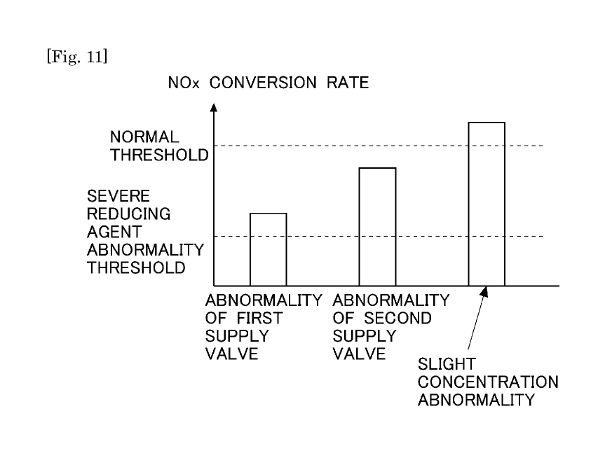

This embodiment, on the other hand, notes a variation in NOx conversion rate in the increase control. FIG. 11 is a graph showing NOx conversion rates in the increase control in the case of abnormality of the first supply valve 41, in the case of abnormality of the second supply valve 42 and in the case of slight concentration abnormality after elapse of a time duration that causes the reducing agent to achieve equilibrium in the first NOx catalyst 31 when the first supply valve 41 is normal, since the instruction time point.

In the case of abnormality of the first supply valve 41, the first supply valve 41 fails to supply the reducing agent to the first NOx catalyst 31, so that the first NOx catalyst 31 fails to convert NOx. The second supply valve 42 is, however, normal, so that the second NOx catalyst 32 works to convert NOx. In this state, an excess of the reducing agent is supplied to the second NOx catalyst 32. The reducing agent accordingly flows out of the second NOx catalyst 32 even before the reducing agent achieves equilibrium in the second NOx catalyst 32. The second NOx sensor 12 detects ammonia other than NOx as described above. The presence of ammonia in the exhaust emission increases the detection value of the second NOx sensor 12. This results in decreasing the NOx conversion rate calculated based on the detection value of the second NOx sensor 12. Accordingly, in the case where the first supply valve 41 is abnormal, the NOx conversion rate of the entire system is relatively low.

In the case of abnormality of the second supply valve 42, on the other hand, the second supply valve 42 fails to supply the reducing agent to the second NOx catalyst 32. The first supply valve 41, however, supplies the reducing agent to the first NOx catalyst 31, so that the first NOx catalyst 31 works to convert NOx. A relatively large amount of the reducing agent is supplied from the first supply valve 41. Accordingly, even before the reducing agent achieves equilibrium in the first NOx catalyst 31, part of the reducing agent supplied from the first supply valve 41 flows out of the first NOx catalyst 31. The flow-out reducing agent is supplied to the second NOx catalyst 32, so that a small amount of NOx is converted in the second NOx catalyst 32. The NOx conversion rate of the entire system is accordingly higher in the case of abnormality of the second supply valve 42 than the NOx conversion rate in the case of abnormality of the first supply valve 41. In the case of abnormality of the second supply valve 42, however, the second NOx catalyst 32 has only a low NOx conversion rate, so that the NOx conversion rate of the entire system is still lower than the normal threshold.

In the case of slight concentration abnormality, a low concentration of the reducing agent is supplied from the first supply valve 41 and the second supply valve 42. This results in supplying the reducing agent to the first NOx catalyst 31 and the second NOx catalyst 32. Even when the reducing agent has a low concentration, increasing the supply amount of the reducing agent to the criterion supply amount causes a sufficient amount of the reducing agent to be eventually supplied to the first NOx catalyst 31 and the second NOx catalyst 32. In the case of slight concentration abnormality, the low concentration of the reducing agent leads to the low NOx conversion rate. Increasing the supply amount of the reducing agent, however, increases the NOx conversion rate. After elapse of a certain time duration since the instruction given to make the supply amount of the reducing agent equal to the criterion supply amount, the NOx conversion rate of the entire system becomes higher than the NOx conversion rate in the case of abnormality of either the first supply valve 41 or the second supply valve 42 and may becomes equal to or higher than the normal threshold.

In the increase control, after elapse of a time duration that causes a distinguishable difference between the NOx conversion rate in the case of slight concentration abnormality and the NOx conversion rate in the case of abnormality of the first supply valve 41 or the second supply valve 42, since the instruction time point, it is identifiable which of slight concentration abnormality and abnormality of the first supply valve 41 or the second supply valve 42, based on the NOx conversion rate at this moment. The time duration that causes a distinguishable difference between the NOx conversion rate in the case of slight concentration abnormality and the NOx conversion rate in the case of abnormality of the first supply valve 41 or the second supply valve 42 may be set to a time duration that causes the reducing agent to achieve equilibrium in the first NOx catalyst 31 when the first supply valve 41 is normal (hereinafter referred to as “slight concentration abnormality determination time duration”). The NOx conversion rate is equal to or higher than the normal threshold in the case of slight concentration abnormality, while being lower than the normal threshold in the case of abnormality of the first supply valve 41 or the second supply valve 42. According to this embodiment, when the NOx conversion rate after elapse of the slight concentration abnormality determination time duration since the instruction time point in the increase control is equal to or higher than the normal threshold, it is determined that slight concentration abnormality occurs and that the first supply valve 41 and the second supply valve 42 are normal. When the NOx conversion rate after elapse of the slight concentration abnormality determination time duration since the instruction time point in the increase control is lower than the normal threshold, on the other hand, it is determined that no slight concentration abnormality occurs and that either the first supply valve 41 or the second supply valve 42 is abnormal. The slight concentration abnormality determination time duration is related to the NOx conversion rate in the regular control, the criterion supply amount, and the amount of the reducing agent adsorbable to the first NOx catalyst 31. These relationships may be determined in advance by experiment, by simulation or the like. The slight concentration abnormality determination time duration of this embodiment corresponds to the second specified time duration of the invention. The slight concentration abnormality determination time duration is shorter than the supply valve abnormality determination time duration.

### <Determination of Abnormality of First Supply Valve 41 and Second Supply Valve 42>

When the abnormality of the system is neither severe reducing agent abnormality nor slight concentration abnormality, either the first supply valve 41 or the second supply valve 42 is abnormal. In this case, it is determinable which of the supply valves 41 and 42 is abnormal in the same manner as described in Embodiment 1.

### <Time Chart in Determination of Abnormality>

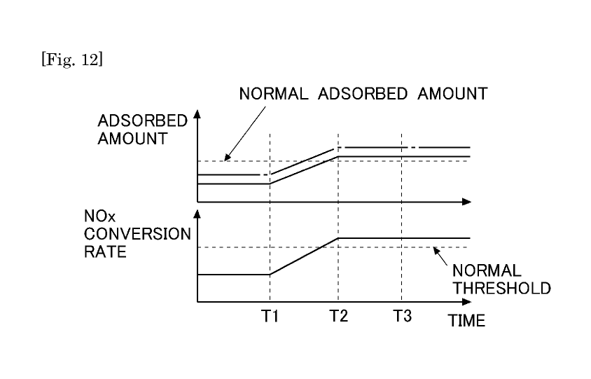

FIG. 12 is a time chart showing one example of variations of the adsorbed amounts of the reducing agent to the respective catalysts and a variation in NOx conversion rate in the case of slight concentration abnormality in the increase control. The time points T1, T2 and T3 in FIG. 12 are identical with the time point T1, T2 and T3 in FIG. 4.

In the case of slight concentration abnormality, before the time point T1, due to insufficiency of the reducing agent, the adsorbed amounts of the reducing agent to the first NOx catalyst 31 and to the second NOx catalyst 32 are smaller than the normal adsorbed amount, and the NOx conversion rate is lower than the normal threshold. In the case of slight concentration abnormality, giving an instruction to make the supply amount of the reducing agent equal to the criterion supply amount causes the supply amount of the reducing agent to approach an adequate amount. This increases the amounts of the reducing agent adsorbed to the first NOx catalyst 31 and to the second NOx catalyst 32. The NOx conversion rate is thus gradually increased after the time point T1. At the time point T2 after elapse of the slight concentration abnormality determination time duration, the reducing agent achieves equilibrium in the first NOx catalyst 31. Almost simultaneously, the reducing agent also achieves equilibrium in the second NOx catalyst 32. After the time point T2, the adsorbed amount of the reducing agent is equal to or larger than the normal adsorbed amount, so that the NOx conversion rate is kept equal to or higher than the normal threshold.

Accordingly, in the case of abnormality of the system other than severe reducing agent abnormality, when the NOx conversion rate is equal to or higher than the normal threshold at the time point T2, it is determined that slight concentration abnormality occurs. In the case of abnormality of the system other than severe reducing agent abnormality, when the NOx conversion rate is lower than the normal threshold at the time point T2 and is equal to or higher than the normal threshold at the time point T3, it is determined that the second supply valve 42 is abnormal. In the case of abnormality of the system other than severe reducing agent abnormality, when the NOx conversion rate is lower than the normal threshold at the time point T2 and is lower than the normal threshold at the time point T3, it is determined that the first supply valve 41 is abnormal.

### <Flow of Abnormality Determination>

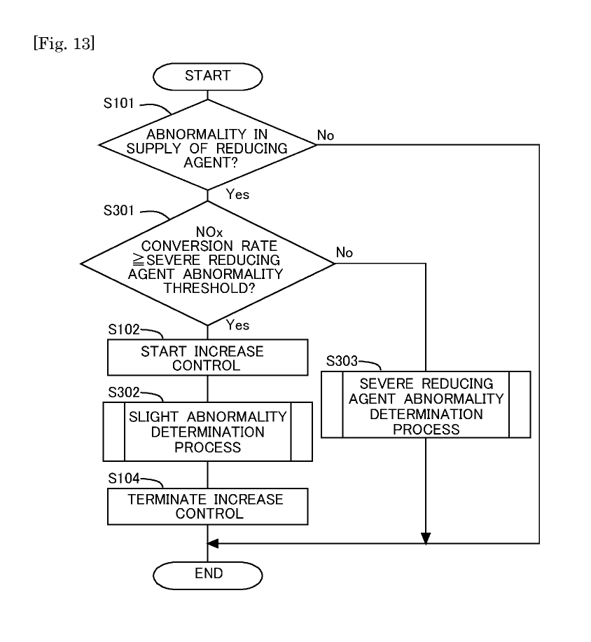

FIG. 13 is a flowchart showing a flow of abnormality determination according to this embodiment. This flow is performed at predetermined time intervals by the ECU 10. The like steps to those of Embodiment 1 described above are expressed by the like step numbers and are not specifically described here.

In the flowchart of FIG. 13, in the case of an affirmative answer at step S101, the flow goes to step S301. At step S301, the ECU 10 determines whether the NOx conversion rate of the entire system is equal to or higher than the severe reducing agent abnormality threshold. The severe reducing agent abnormality threshold may be determined in advance by experiment, by simulation or the like. At step S301, it is determined whether severe reducing agent abnormality occurs. The NOx conversion rate is lower in the case of severe reducing agent abnormality than the NOx conversion rate in the case of slight abnormality. Accordingly, the presence or the absence of severe reducing agent abnormality is detectable by comparison between the NOx conversion rate and the severe reducing agent abnormality threshold. This flow first detects the presence or the absence of severe reducing agent abnormality in the regular control. In the case of an affirmative answer at step S301, it is determined that no severe reducing agent abnormality occurs, and the flow goes to step S102. In the case of a negative answer at step S301, on the other hand, it is determined that severe reducing agent abnormality occurs, and the flow goes to step S303.

After starting the increase control at step S102, the flow goes to step S302. At step S302, the ECU 10 performs a slight abnormality determination process or more specifically identifies which of slight concentration abnormality, abnormality of the first supply valve 41 and abnormality of the second supply valve 42. This slight abnormality determination process will be described later in detail. After conclusion of step S302, the flow goes to step S104.

At step S303, on the other hand, the ECU 10 performs a severe reducing agent abnormality determination process or more specifically identifies which of abnormality that no reducing agent is stored in the urea tank 43, severe concentration abnormality and abnormality that the concentration of the reducing agent is 0%. This severe reducing agent abnormality determination process will be described later in detail. After conclusion of step S303, this flow is terminated.

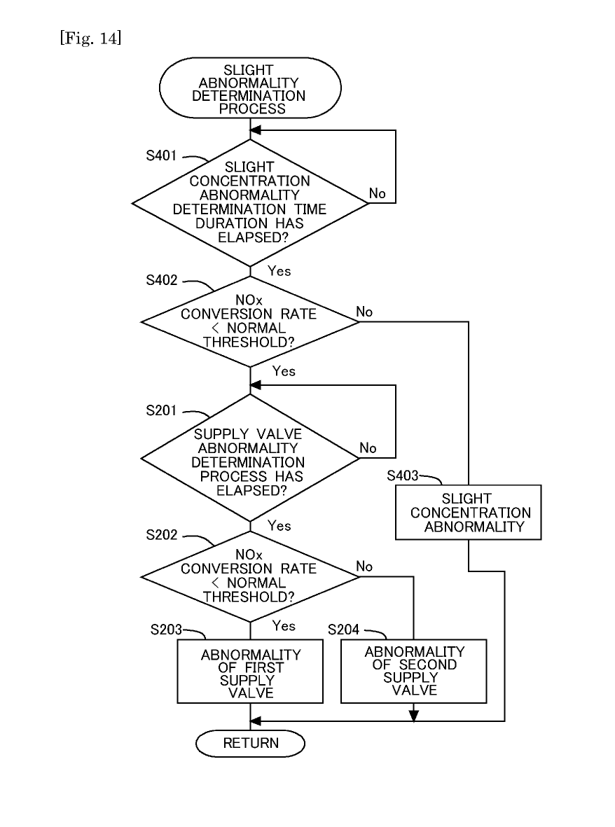

The following describes the slight abnormality determination process performed at step S302. FIG. 14 is a flowchart showing the slight abnormality determination process. The like steps to those of Embodiment 1 described above are expressed by the like step numbers and are not specifically described here.

At step S401, the ECU 10 determines whether the slight concentration abnormality determination time duration has elapsed since the instruction time point of the increase control at step S102. More specifically, the ECU 10 determines whether a time duration that allows for determination of the presence or the absence of slight concentration abnormality has elapsed. In the case of an affirmative answer at step S401, the flow goes to step S402. In the case of a negative answer at step S401, on the other hand, the flow repeats step S401.

At step S402, the ECU 10 determines whether the NOx conversion rate at the current moment is lower than the normal threshold. More specifically, it is determined whether the NOx conversion rate after elapse of the slight concentration abnormality determination time duration has elapsed since the instruction time point in the increase control is lower than the normal threshold. The normal threshold may be determined in advance by experiment, by simulation or the like as the lower limit value of the NOx conversion rate in the normal state of the system. After elapse of the slight concentration abnormality determination time duration, the NOx conversion rate becomes equal to or higher than the normal threshold in the case of slight concentration abnormality, while being lower than the normal threshold in the case of abnormality of the first supply valve 41 or the second supply valve 42. Accordingly, the determination result of this step identifies which of slight concentration abnormality and abnormality of the first supply valve 41 or the second supply valve 42. In the case of an affirmative answer at step S402, it is determined that either the first supply valve 41 or the second supply valve 42 is abnormal, and the flow goes to step S201. In the case of a negative answer at step S402, on the other hand, the flow goes to step S403 to determine that slight concentration abnormality occurs. Subsequently, this flow is terminated, and the slight abnormality determination process of step S302 is terminated.

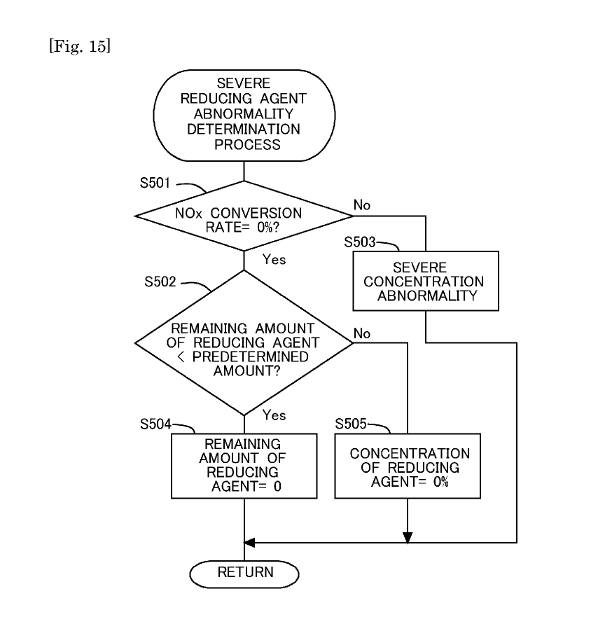

The following describes the severe reducing agent abnormality determination process performed at step S303. FIG. 15 is a flowchart showing the severe reducing agent abnormality determination process.

At step S501, the ECU 10 determines whether the NOx conversion rate is 0%. In other words, it is determined whether NOx is not at all converted. In the case of an affirmative answer at step S501, the flow goes to step S502. In the case of a negative answer at step S501, on the other hand, the NOx conversion rate is low but is not 0%, so that the presence of the reducing agent is suggested. Accordingly, the flow goes to step S503 to determine that severe concentration abnormality occurs.

At step S502, the ECU 10 determines whether the remaining amount of the reducing agent stored in the urea tank 43 is less than a predetermined amount. The predetermined amount is a lower limit value of the remaining amount of the reducing agent suppliable from the first supply valve 41 and the second supply valve 42. The remaining amount of the reducing agent is detected by the reducing agent quantity sensor 46. In other words, it is determined whether the reducing agent is used up at this step. According to one modification, the ECU 10 may determine whether the remaining amount of the reducing agent is zero at step S502.

In the case of an affirmative answer at step S502, the flow goes to step S504 to determine that no urea water is stored in the urea tank 43 or, in other words, that the remaining amount of the reducing agent is zero. In the case of a negative answer at step S502, on the other hand, the flow goes to step S505 to determine that the concentration of the reducing agent is 0%. When the NOx conversion rate is 0%, it is expected that no reducing agent is supplied. The cause of no supply of the reducing agent is attributable to the case that the remaining amount of the reducing agent is zero or to the case that the concentration of the reducing agent is 0%. The determination of step S502 identifies the cause of the abnormality.

As described above, the flow of this embodiment first determines whether the abnormality is severe reducing agent abnormality or slight abnormality. In the case of slight abnormality, the flow subsequently determines whether the slight abnormality is slight concentration abnormality or abnormality of either the first supply valve 41 or the second supply valve 42. In the case of abnormality of either the first supply valve 41 or the second supply valve 42, the flow further determines whether the first supply valve 41 is abnormal or the second supply valve 42 is abnormal. The flow of this embodiment obtains the NOx conversion rate at a predefined timing and identifies the type of abnormality based on the obtained NOx conversion rate. According to one modification, the NOx conversion rate obtained at the predefined timing may be stored, and the abnormality determination may be performed at any time, based on the stored NOx conversion rate.

As described above, when abnormality occurs in supply of the reducing agent, this embodiment identifies the type of the abnormality among severe reducing agent abnormality (more specifically, abnormality that no reducing agent is stored in the urea tank 43, severe concentration abnormality and abnormality that the concentration of the reducing agent is 0%), slight concentration abnormality, abnormality of the first supply valve 41 and abnormality of the second supply valve 42.

### Embodiment 3

This embodiment describes the case where the first supply valve 41 and the second supply valve 42 are deteriorated simultaneously. The configuration of the other devices, components and the like is identical with that of Embodiment 1 and is not specifically described here. This embodiment uses the reducing agent concentration sensor 47.

Simultaneous deterioration of the first supply valve 41 and the second supply valve 42 is unlikely to occur, but the first supply valve 41 and the second supply valve 42 may deteriorate over time simultaneously. This embodiment describes the case of simultaneous deterioration of the first supply valve 41 and the second supply valve 42. Such deterioration includes the case where the NOx conversion rate calculated in the regular control is equal to or higher than the severe reducing agent abnormality threshold but is lower than the normal threshold and the case where the calculated NOx conversion rate is lower than the severe reducing agent abnormality threshold. Deterioration that causes the NOx conversion rate to be lower than the severe reducing agent abnormality threshold is called severe supply valve deterioration. Deterioration that causes the NOx conversion rate to be equal to or higher than the severe reducing agent abnormality threshold but lower than the normal threshold is called slight deterioration. The description of this embodiment is on the assumption that the first supply valve 41 and the second supply valve 42 have comparable levels of deterioration. In the slight deterioration or severe supply valve deterioration, the supply amounts of the reducing agent per unit time by the first supply valve 41 and the second supply valve 42 are reduced from the supply amounts in the normal state. At the maximum level of deterioration, both the first supply valve 41 and the second supply valve 42 fail to supply the reducing agent.

The severe reducing agent abnormality threshold denotes a NOx conversion rate in the regular control at a lower limit value of the concentration of the reducing agent that is likely to increase the NOx conversion rate to the normal threshold in the increase control as described above. The slight deterioration and the severe supply valve deterioration are defined as described above by taking into account this severe reducing agent abnormality threshold. In the case of slight deterioration, the NOx conversion rate is lower than the normal threshold in the regular control but becomes equal to or higher than the normal threshold in the increase control. In the case of severe supply valve deterioration, on the other hand, the NOx conversion rate does not reach the normal threshold even in the increase control. The relationship between the slight deterioration and the severe supply valve deterioration may be regarded like the relationship between the slight concentration abnormality and the severe concentration abnormality.

In the case of slight deterioration of the first supply valve 41 and the second supply valve 42, the NOx conversion rate of the entire system is lower than the normal threshold even when the regular control is performed to supply the reducing agent from the first supply valve 41 and the second supply valve 42 according to the amount of NOx discharged from the internal combustion engine 1. In other words, in the case of simultaneous slight deterioration of the first supply valve 41 and the second supply valve 42, the NOx conversion rate is decreased in both the first NOx catalyst 31 and the second NOx catalyst 32. In this case, however, the reducing agent is still suppliable. Giving an instruction to make the supply amount of the reducing agent equal to the criterion supply amount enables the NOx conversion rate to increase in the first NOx catalyst 31 and in the second NOx catalyst 32. In the increase control, after elapse of a time duration that causes a distinguishable difference between the NOx conversion rate in the case of slight deterioration and the NOx conversion rate in the case of abnormality of the first supply valve 41 or the second supply valve 42, since the instruction time point, it is identifiable which of slight deterioration and abnormality of the first supply valve 41 or the second supply valve 42, based on the NOx conversion rate at this moment. The time duration that causes a distinguishable difference between the NOx conversion rate in the case of slight deterioration and the NOx conversion rate in the case of abnormality of the first supply valve 41 or the second supply valve 42 may be set to, for example, the slight concentration abnormality determination time duration described above. In the case of slight deterioration of the first supply valve 41 and the second supply valve 42, the NOx conversion rate of the entire system is lower than the normal threshold in the regular control, and becomes equal to or higher than the normal threshold after elapse of the slight concentration abnormality determination time duration since the instruction time point in the increase control. The NOx conversion rate, however, shows a similar variation in the case of slight concentration abnormality. This embodiment identifies which of slight deterioration of the first supply valve 41 and the second supply valve 42 and slight concentration abnormality. The slight concentration abnormality determination time duration of this embodiment corresponds to the third specified time duration of the invention.

This embodiment uses the reducing agent concentration sensor 47 to determine whether the abnormality is slight concentration abnormality. More specifically, when the NOx conversion rate of the entire system is equal to or higher than the normal threshold after elapse of the slight concentration abnormality determination time duration since the instruction time point in the increase control, the abnormality may be slight concentration abnormality or slight deterioration of the first supply valve 41 and the second supply valve 42. When the reducing agent concentration detected by the reducing agent concentration sensor 47 is within a range of slight concentration abnormality, the abnormality is determined not as slight deterioration of the first supply valve 41 and the second supply valve 42 but as slight concentration abnormality. In other words, when the reducing agent concentration detected by the reducing agent concentration sensor 47 is within a normal range, the NOx conversion rate of the entire system is lower than the normal threshold in the regular control and is equal to or higher than the normal threshold after elapse of the slight concentration abnormality determination time duration since the instruction time point in the increase control, it is determined that slight deterioration occurs in the first supply valve 41 and the second supply valve 42.

In the case of severe supply valve deterioration of the first supply valve 41 and the second supply valve 42, on the other hand, the NOx conversion rate is lower than the severe reducing agent abnormality threshold in the regular control. A comparable level of the NOx conversion rate may be obtained in the case of severe reducing agent abnormality, but the severe reducing agent abnormality is identifiable by using the reducing agent concentration sensor 47. Accordingly, when the detection value of the reducing agent concentration sensor 47 suggests no likelihood of severe reducing agent abnormality and the NOx conversion rate is lower than the severe reducing agent abnormality threshold in the regular control, it is determined that severe supply valve deterioration occurs in the first supply valve 41 and the second supply valve 42.

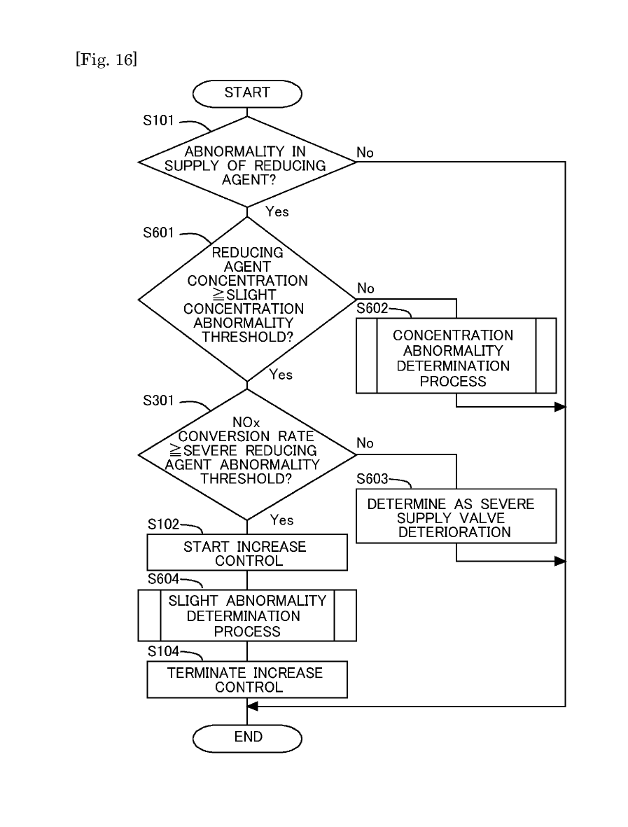

FIG. 16 is a flowchart showing a flow of abnormality determination according to this embodiment. This flow is performed at predetermined time intervals by the ECU 10. The like steps to those of Embodiment 1 or Embodiment 2 described above are expressed by the like step numbers and are not specifically described here.

In the flowchart of FIG. 16, in the case of an affirmative answer at step S101, the flow goes to step S601. At step S601, the ECU 10 determines whether the reducing agent concentration is equal to or higher than the slight concentration abnormality threshold. The slight concentration abnormality threshold is a lower limit value of the reducing agent concentration in the case where no slight concentration abnormality occurs in the reducing agent. The reducing agent concentration is detected by the reducing agent concentration sensor 47. When the remaining amount of the reducing agent is insufficient, the detected reducing agent concentration is 0%. It is determined whether the reducing agent is abnormal at step S601. More specifically, when the reducing agent concentration is equal to or higher than the slight concentration abnormality threshold, it is determined that neither slight concentration abnormality nor severe reducing agent abnormality occurs. In the case of an affirmative answer at step S601, the flow goes to step S301. In the case of a negative answer at step S601, on the other hand, the flow goes to step S602 to perform a concentration abnormality determination process. The concentration abnormality determination process will be described later in detail.

In the flowchart of FIG. 16, in the case of a negative answer at step S301, the flow goes to step S603 to determine that severe supply valve deterioration occurs. More specifically, the NOx conversion rate of lower than the severe reducing agent abnormality threshold despite no abnormality of the reducing agent is attributable to a failure in supply of the reducing agent from the first supply valve 41 and the second supply valve 42. Accordingly, it is determined that severe supply valve deterioration occurs in the first supply valve 41 and the second supply valve 42 at step S603.

In the flowchart of FIG. 16, after starting the increase control at step S102, the flow goes to step S604. At step S604, the ECU 10 performs a slight abnormality determination process or more specifically identifies which of abnormality of the first supply valve 41, abnormality of the second supply valve 42 and slight deterioration of both the first supply valve 41 and the second supply valve 42. This slight abnormality determination process will be described later in detail. After conclusion of step S604, the flow goes to step S104.

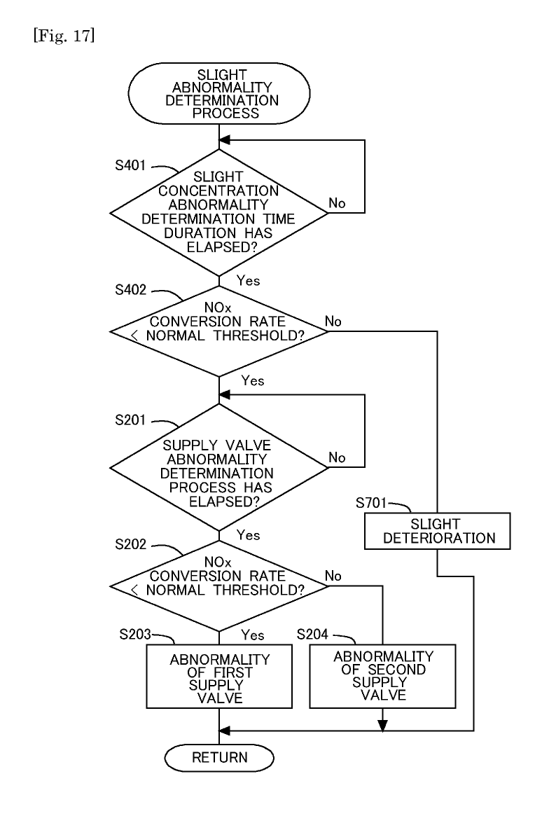

FIG. 17 is a flowchart showing the slight abnormality determination process performed at step S604. The like steps to those of Embodiment 1 or Embodiment 2 described above are expressed by the like step numbers and are not specifically described here.

In the flowchart of FIG. 17, in the case of a negative answer at step S402, the flow goes to step S701 to determine that slight deterioration occurs. In the flowchart of FIG. 14, in the case of a negative answer at step S402, it is determined that slight concentration abnormality occurs. In the flowchart of FIG. 17, however, it has already been determined that slight concentration abnormality is unlikely to occur at step S601. Even in the case of slight deterioration of the first supply valve 41 and the second supply valve 42, the reducing agent is still suppliable from the first supply valve 41 and the second supply valve 42. The NOx conversion rate thus becomes equal to or higher than the normal threshold after elapse of the slight concentration abnormality determination time duration. Accordingly, when the NOx conversion rate is equal to or higher than the normal threshold after elapse of the slight concentration abnormality determination time duration, it is determined that slight deterioration occurs in the first supply valve 41 and the second supply valve 42.

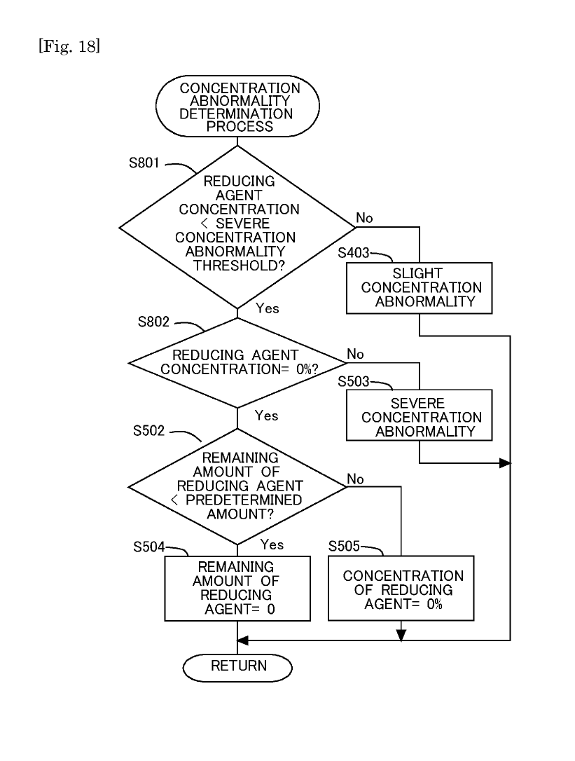

FIG. 18 is a flowchart showing the concentration abnormality determination process performed at step S602. The like steps to those of Embodiment 2 described above are expressed by the like step numbers and are not specifically described here.

At step S801, the ECU 10 determines whether the reducing agent concentration detected by the reducing agent concentration sensor 47 is lower than a severe concentration abnormality threshold. The severe concentration abnormality threshold is a lower limit value of the concentration of the reducing agent in the case where no severe concentration abnormality occurs in the reducing agent and may be determined in advance by experiment, by simulation or the like. In other words, it is determined whether severe reducing agent abnormality occurs. In the case of an affirmative answer at step S801, it is determined that severe reducing agent abnormality occurs, and the flow goes to step S802. In the case of a negative answer at step S801, on the other hand, it is determined that severe reducing agent abnormality does not occur. It has, however, been determined at step S601 that at least concentration abnormality of the reducing agent occurs. In the case of a negative answer at step S801, the flow accordingly goes to step S403 to determine that slight concentration abnormality occurs.

At step S802, the ECU 10 determines whether the reducing agent concentration is 0%. The determination of step S802 may be replaced by determination of whether the NOx conversion rate is 0% like step S501 described above. In other words, it is determined at step S802 whether NOx is not at all converted. In the case of an affirmative answer at step S802, the flow goes to step S502. In the case of a negative answer at step S802, on the other hand, the flow goes to step S503 to determine that severe concentration abnormality occurs.

As described above, this embodiment enables simultaneous deterioration of the first supply valve 41 and the second supply valve 42 to be detected with high accuracy.

### Embodiment 4

This embodiment describes the case of occurrence of abnormality that provides a higher gain of the second NOx sensor 12 than the actual value. In the description below, providing a higher gain of the second NOx sensor 12 than the actual value is called “gain deviation”. The description of this embodiment is on the assumption that multiple abnormalities do not occur simultaneously in the first supply valve 41, the second supply valve 42 and the reducing agent. The configuration of the other devices, components and the like is identical with that of Embodiment 1 and is not specifically described here.

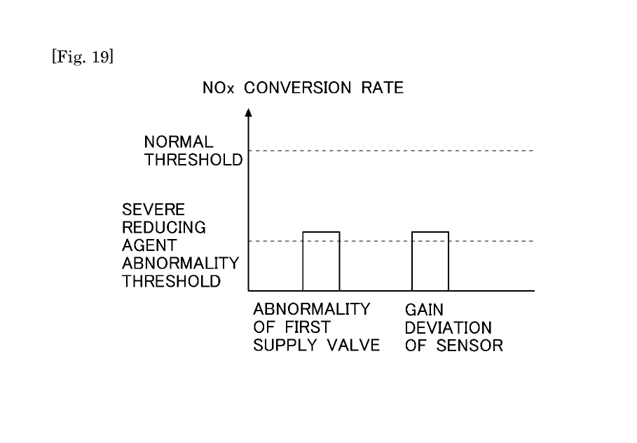

FIG. 19 is a graph showing the relationship between the NOx conversion rate and the severe reducing agent abnormality threshold in the regular control, in the case of abnormality of the first supply valve 41 and in the case of gain deviation of the second NOx sensor 12. In the case of gain deviation of the second NOx sensor 12, the detected NOx concentration becomes higher than the actual NOx concentration, so that the calculated NOx conversion rate becomes lower than the actual NOx conversion rate. This results in a low NOx conversion rate calculated in the regular control. Comparable levels of the NOx conversion rate may be obtained in the regular control in the case of gain deviation of the second NOx sensor 12 and in the case of abnormality of the first supply valve 41. In this case, the NOx conversion rate may be equal to or higher than the severe reducing agent abnormality threshold and lower than the normal threshold in either abnormality. Additionally, even after the instruction time point in the increase control, the NOx conversion rate may be equal to or higher than the severe reducing agent abnormality threshold and lower than the normal threshold in either case of abnormality of the first supply valve 41 or gain deviation of the second NOx sensor 12. Accordingly it is difficult to distinguish the case of gain deviation of the second NOx sensor 12 from the case of abnormality of the first supply valve 41 by comparison between the severe reducing agent abnormality threshold and the NOx conversion rate. This embodiment is thus configured to identify which of gain deviation of the second NOx sensor 12 and abnormality of the first supply valve 41.

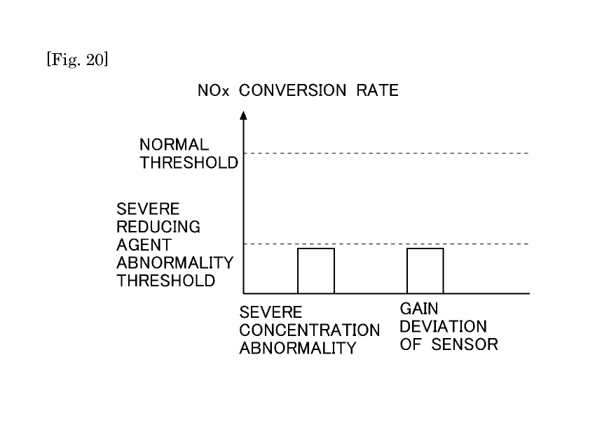

FIG. 20 is a graph showing the relationship between the NOx conversion rate and the severe reducing agent abnormality threshold in the regular control, in the case of severe concentration abnormality and in the case of gain deviation of the second NOx sensor 12. It is on the premise that the degree of gain deviation of the second NOx sensor 12 in FIG. 20 is greater than the degree of gain deviation in FIG. 19. In the case of gain deviation of the second NOx sensor 12, the NOx concentration detected in the regular control becomes higher than the actual NOx concentration, so that the NOx conversion rate calculated based on the detection value of the second NOx sensor 12 becomes lower than the actual NOx conversion rate. This may result in the lower NOx conversion rate calculated in the regular control than the severe reducing agent abnormality threshold. In the case of severe concentration abnormality, on the other hand, the NOx conversion rate may also be lower than the severe reducing agent abnormality threshold in the regular control. Accordingly, when the NOx conversion rate is higher than zero and lower than the severe reducing agent abnormality threshold in the regular control, the cause may be attributed to either abnormality of the reducing agent or gain deviation of the second NOx sensor 12. It is, however, difficult to distinguish gain deviation of the second NOx sensor 12 from abnormality of the reducing agent by comparison between their NOx conversion rates in the regular control. This embodiment is thus configured to identify which of gain deviation of the second NOx sensor 12 and severe concentration abnormality.

After the instruction time point in the increase control, a difference is made between the calculated NOx conversion rates in the case of gain deviation of the second NOx sensor 12 and in the case of abnormality of the first supply valve 41. Accordingly, in the increase control, after elapse of a time duration that causes a distinguishable difference between the NOx conversion rate in the case of gain deviation of the second NOx sensor 12 and the NOx conversion rate in the case of abnormality of the first supply valve 41, since the instruction time point, it is identifiable which of gain deviation of the second NOx sensor 12 and abnormality of the first supply valve 41, based on the NOx conversion rate at this moment. For example, in the case where the reducing agent achieves equilibrium in the second NOx catalyst 32, the reducing agent flows out of the second NOx catalyst 32, so as to increase the detection value of the second NOx sensor 12. This results in making a distinguishable difference between the NOx conversion rates in the case of gain deviation of the second NOx sensor 12 and in the case of abnormality of the first supply valve 41, for example, after elapse of the supply valve abnormality determination time duration since the instruction time point in the increase control. The supply valve abnormality determination time duration of this embodiment corresponds to the fourth specified time duration of the invention.

Similarly, after the instruction time point in the increase control, a difference is made between the calculated NOx conversion rates in the case of gain deviation of the second NOx sensor 12 and in the case of severe concentration abnormality. Accordingly, in the increase control, after elapse of a time duration that causes a distinguishable difference between the NOx conversion rate in the case of gain deviation of the second NOx sensor 12 and the NOx conversion rate in the case of severe concentration abnormality, since the instruction time point, it is identifiable which of gain deviation of the second NOx sensor 12 and severe concentration abnormality, based on the NOx conversion rate at this moment. For example, in the case of gain deviation of the sensor, after elapse of the slight concentration abnormality determination time duration since the instruction time point in the increase control, the reducing agent flows out of the second NOx catalyst 32, so as to increase the detection value of the second NOx sensor 12. This results in making a distinguishable difference between the NOx conversion rates in the case of gain deviation of the second NOx sensor 12 and in the case of severe concentration abnormality, for example, after elapse of the slight concentration abnormality determination time duration since the instruction time point in the increase control. The slight concentration abnormality determination time duration of this embodiment corresponds to the fifth specified time duration of the invention.

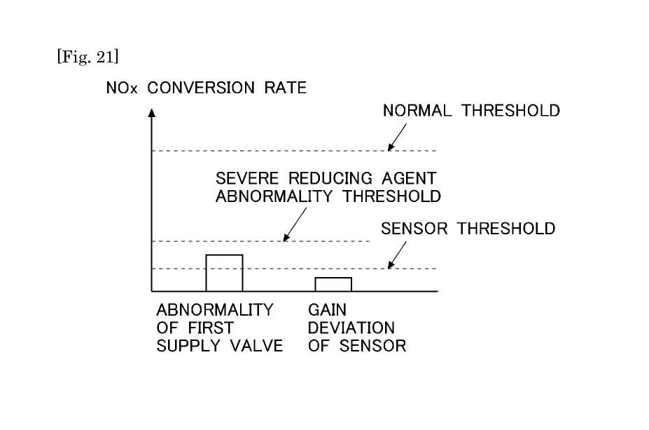

FIG. 21 is a graph showing the relationship between the NOx conversion rate and the severe reducing agent abnormality threshold after elapse of the supply valve abnormality determination time duration since the instruction time point in the increase control, in the case of abnormality of the first supply valve 41 and in the case of gain deviation of the second NOx sensor 12. FIG. 21 shows the results when an instruction is given to make the supply amount of the reducing agent equal to the criterion supply amount under the relationship of FIG. 19.

In the case of gain deviation of the second NOx sensor 12, neither the reducing agent supply device 4 nor the reducing agent is abnormal. Giving an instruction to make the supply amount of the reducing agent equal to the criterion supply amount accordingly results in an excess of the reducing agent in the first NOx catalyst 31 and in the second NOx catalyst 32. The reducing agent (ammonia) thus flows out of the first NOx catalyst 31 and the second NOx catalyst 32. Detection of ammonia by the second NOx sensor 12 increases the detection value of the second NOx sensor 12. This results in decreasing the calculated NOx conversion rate. In the case of abnormality of the first supply valve 41, on the other hand, no reducing agent is supplied from the first supply valve 41. Even when an instruction is given to make the supply amount of the reducing agent equal to the criterion supply amount, no reducing agent flows out of the first NOx catalyst 31. The amount of the reducing agent supplied to the second NOx catalyst 32 in the case of abnormality of the first supply valve 41 is accordingly less than the amount of the reducing agent supplied to the second NOx catalyst 32 in the case of gain deviation of the second NOx sensor 12. The amount of the reducing agent flowing out of the second NOx catalyst 32 in the case of abnormality of the first supply valve 41 is thereby less than the amount of the reducing agent flowing out of the second NOx catalyst 32 in the case of gain deviation of the second NOx sensor 12. As a result, the NOx conversion rate calculated based on the detection value of the second NOx sensor 12 in the case of gain deviation of the second NOx sensor 12 is lower than the NOx conversion rate in the case of abnormality of the first supply valve 41. In the case of abnormality of the second supply valve 42, when an instruction is given to make the supply amount of the reducing agent equal to the criterion supply amount, the reducing agent flowing out of the first NOx catalyst 31 is adsorbed to the second NOx catalyst 32, so that the second NOx catalyst 32 also works to convert NOx. This makes the NOx conversion rate of the entire system equal to or higher than the normal threshold. Accordingly, the NOx conversion rate after elapse of the supply valve abnormality determination time duration in the case of abnormality of the second supply valve 42 is higher than the NOx conversion rates in the case of abnormality of the first supply valve 41 and in the case of gain deviation of the second NOx sensor 12.

A lower limit value of the NOx conversion rate after elapse of the supply valve abnormality determination time duration in the case where the second NOx sensor 12 has no gain deviation is accordingly set to a sensor threshold. When the NOx conversion rate after elapse of the supply valve abnormality determination time duration is equal to or higher than the sensor threshold, it is determined that the first supply valve 41 is abnormal. When the NOx conversion rate is lower than the sensor threshold, on the other hand, it is determined that the second NOx sensor 12 has gain deviation. In the case where the second NOx sensor 12 is normal, the NOx conversion rate reaches its minimum when all the reducing agent other than the amount used for conversion of NOx flows out of the second NOx catalyst 32. This minimum NOx conversion rate may alternatively be set to the sensor threshold. The gain deviation of the second NOx sensor 12 is affected by the NOx concentration in the exhaust emission flowing into the first NOx catalyst 31 and the instruction value of the supply amount of the reducing agent. The sensor threshold may thus be set based on the NOx concentration in the exhaust emission flowing into the first NOx catalyst 31 and the instruction value of the supply amount of the reducing agent. The relationship of the sensor threshold to the NOx concentration in the exhaust emission flowing into the first NOx catalyst 31 and the instruction value of the supply amount of the reducing agent may be specified in advance by experiment or the like.

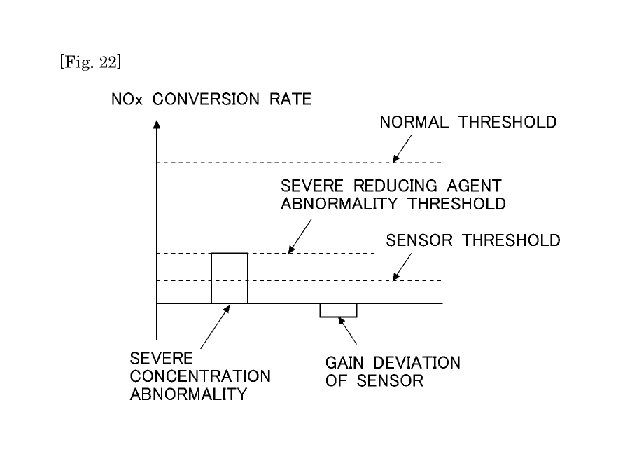

FIG. 22 is a graph showing the relationship between the NOx conversion rate and the severe reducing agent abnormality threshold after elapse of the slight concentration abnormality determination time duration since the instruction time point in the increase control, in the case of severe concentration abnormality and in the case of gain deviation of the second NOx sensor 12. FIG. 22 shows the results when an instruction is given to make the supply amount of the reducing agent equal to the criterion supply amount under the relationship of FIG. 20.

As described above, in the case of gain deviation of the second NOx sensor 12, a large amount of the reducing agent flows out of the second NOx catalyst 32 after elapse of the slight concentration abnormality determination time duration since the instruction time point in the increase control. Since both the first supply valve 41 and the second supply valve 42 are normal, giving an instruction to make the supply amount of the reducing agent equal to the criterion supply amount causes an excess amount of the reducing agent to be supplied from both the first supply valve 41 and the second supply valve 42. The second NOx sensor 12 detects ammonia other than NOx. The presence of ammonia in the exhaust emission accordingly increases the detection value of the second NOx sensor 12. This results in decreasing the NOx conversion rate calculated based on the detection value of the second NOx sensor 12. A large amount of the reducing agent (ammonia) flows out of the second NOx catalyst 32 after elapse of the slight concentration abnormality determination time duration since the instruction time point in the increase control. This results in decreasing the calculated NOx conversion rate in the case of gain deviation of the second NOx sensor 12. In the illustrated example of FIG. 22, the NOx conversion rate decreases to a negative value.

In the case of severe concentration abnormality, the adsorbed amount of the reducing agent is also increased after elapse of the slight concentration abnormality determination time duration since the instruction time point in the increase control. In the case of severe concentration abnormality, however, the original adsorbed amount of the reducing agent is a low level. Even when the adsorbed amount of the reducing agent is increased, the reducing agent is unlikely to flow out of the second NOx catalyst 32. Additionally, an increase in amount of the reducing agent adsorbed to both the first and second NOx catalysts 31 and 32 recovers the NOx conversion rates in both the first and the second NOx catalysts 31 and 32. This results in increasing the calculated NOx conversion rate. As a result, the NOx conversion rate calculated after elapse of the slight concentration abnormality determination time duration since the instruction time point in the increase control is increased in the case of severe concentration abnormality, while being decrease in the case of gain deviation of the second NOx sensor 12. Gain deviation of the second NOx sensor 12 and severe concentration abnormality are thus distinguishable from each other, based on a change in NOx conversion rate in the regular control and after elapse of the slight concentration abnormality determination time duration since the instruction time point in the increase control. The calculated NOx conversion rate in the case of gain deviation of the second NOx sensor 12 is lower than the calculated NOx conversion rate in the case of severe concentration abnormality. When the NOx conversion rate is equal to or higher than the sensor threshold, it is determined that severe concentration abnormality occurs. When the NOx conversion rate is lower than the sensor threshold, on the other hand, it is determined that the second NOx sensor 12 has gain deviation.

As described above, when the NOx conversion rate after elapse of the slight concentration abnormality determination time duration since the instruction time point in the increase control is lower than the sensor threshold, it is determined that the second NOx sensor 12 has gain deviation. When the NOx conversion rate is equal to or higher than the sensor threshold, on the other hand, it is determined that severe concentration abnormality occurs. Severe concentration abnormality and abnormality of the first supply valve 41 are distinguishable from each other by the configuration of the embodiment described above.

As described above, in the case of sensor gain of the second NOx sensor 12, the amount of the reducing agent flowing out of the second NOx catalyst 32 increases with an increase in supply amount of the reducing agent. This results in decreasing the calculated NOx conversion rate. In the case of severe concentration abnormality, on the other hand, an increase in supply amount of the reducing agent recovers the NOx conversion rates in the first NOx catalyst 31 and the second NOx catalyst 32. This results in increasing the NOx conversion rate. Accordingly, it may be determined that the second NOx sensor 12 has gain deviation in the case of a decrease in NOx conversion rate after the instruction time point in the increase control. It may be determined that severe concentration abnormality occurs, on the other hand, in the case of an increase in NOx conversion rate after the instruction time point in the increase control.

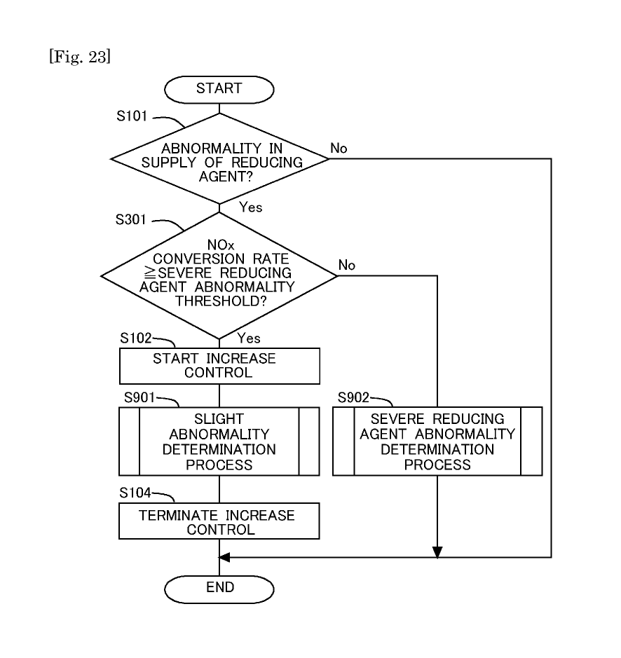

FIG. 23 is a flowchart showing a flow of abnormality determination according to this embodiment. This flow is performed at predetermined time intervals by the ECU 10. The like steps to those of Embodiment 1 or Embodiment 2 described above are expressed by the like step numbers and are not specifically described here.

In the flowchart of FIG. 23, after conclusion of step S102, the flow goes to step S901. At step S901, the ECU 10 performs a slight abnormality determination process or more specifically identifies which of slight concentration abnormality, abnormality of the first supply valve 41, abnormality of the second supply valve 42 and gain deviation of the second NOx sensor 12. This slight abnormality determination process will be described later in detail. After conclusion of step S901, the flow goes to step S104.

In the flowchart of FIG. 23, in the case of a negative answer at step S301, the flow goes to step S902. At step S902, the ECU 10 performs a severe reducing agent abnormality determination process or more specifically identifies which of abnormality that no reducing agent is stored in the urea tank 43, severe concentration abnormality, abnormality that the concentration of the reducing agent is 0% and gain deviation of the second NOx sensor 12. This severe reducing agent abnormality determination process will be described later in detail. After conclusion of step S902, this flow is terminated.

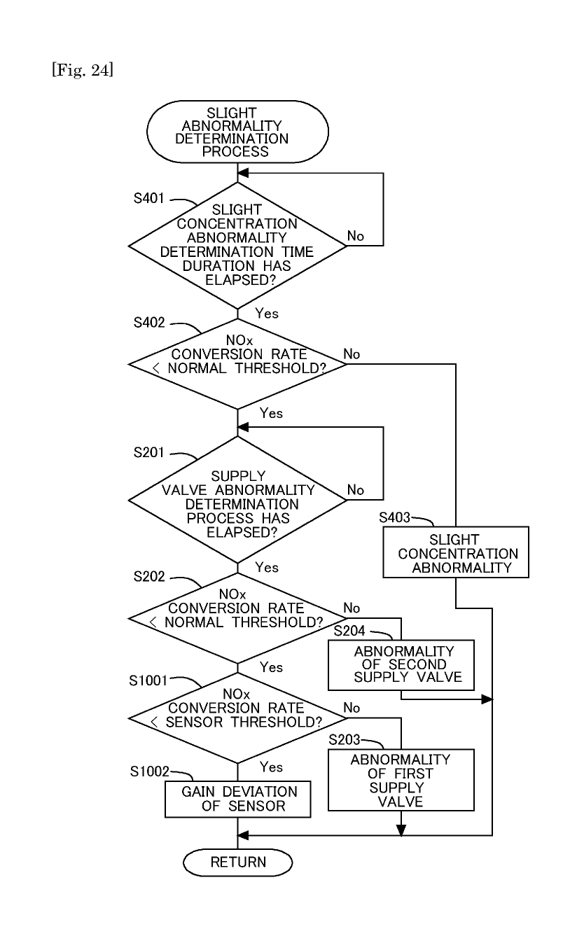

FIG. 24 is a flowchart showing the slight abnormality determination process performed at step S901. The like steps to those of Embodiment 1 or Embodiment 2 described above are expressed by the like step numbers and are not specifically described here.

At step S1001, the ECU 10 determines whether the NOx conversion rate at the current moment is lower than the sensor threshold. More specifically, the NOx conversion rate after elapse of the supply valve abnormality determination time duration since the instruction time point in the increase control is lower than the sensor threshold. The sensor threshold may be determined in advance by experiment, by simulation or the like. After elapse of the supply valve abnormality determination time duration, the NOx conversion rate is equal to or higher than the normal threshold in the case where the second supply valve 42 is abnormal, while being lower than the normal threshold in the case where the first supply valve 41 is abnormal. In the case of gain deviation of the second NOx sensor 12, the NOx conversion rate is lower than the normal threshold after elapse of the supply valve abnormality determination time duration. Accordingly, in the case of an affirmative answer at step S202, the cause is attributed to either abnormality of the first supply valve 41 or gain deviation of the second NOx sensor 12. The determination of step S1001 then identifies which of abnormality of the first supply valve 41 and gain deviation of the second NOx sensor 12.

In the case of an affirmative answer at step S1001, the flow goes to step S1002 to determine that the second NOx sensor 12 has gain deviation. In the case of a negative answer at step S1001, on the other hand, the flow goes to step S203 to determine that the first supply valve 41 is abnormal. Subsequently, this flow is terminated, and the slight abnormality determination process of step S901 is terminated.

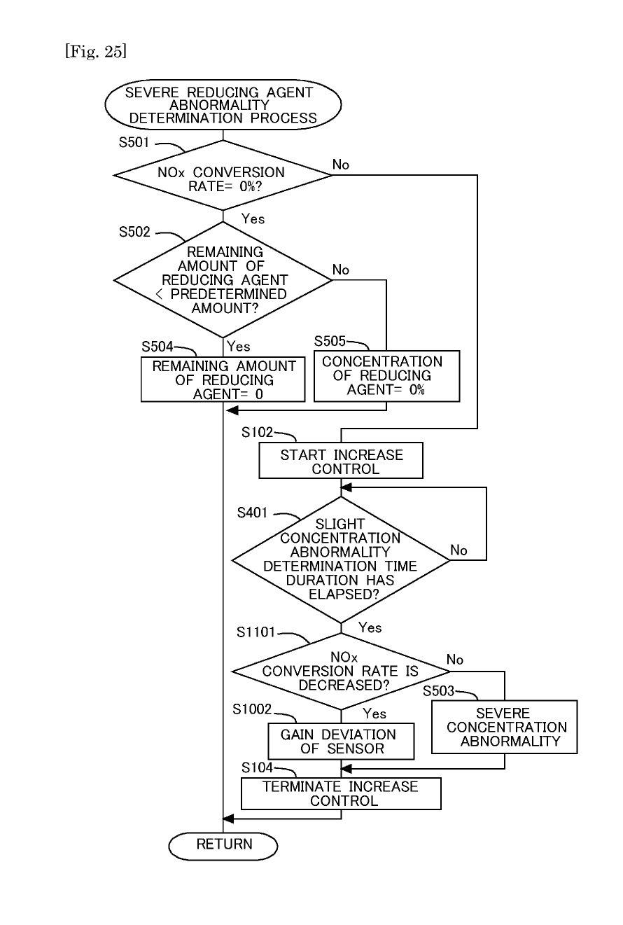

FIG. 25 is a flowchart showing the severe reducing agent abnormality determination process performed at step S902. The like steps to those of Embodiment 2 described above are expressed by the like step numbers and are not specifically described here.

In the flowchart of FIG. 25, in the case of a negative answer at step S501, the flow goes to step S102. When the NOx conversion rate is not 0% but is lower than the severe reducing agent abnormality threshold, the cause is attributed to either severe concentration abnormality or gain deviation of the second NOx sensor 12. Accordingly, a series of processing of and after step S102 is performed to identify which of severe concentration abnormality and gain deviation of the second NOx sensor 12. At step S102, the ECU 10 starts increasing the supply amount of the reducing agent or more specifically gives an instruction to make the supply amount of the reducing agent equal to the criterion supply amount. As shown in FIG. 22, after elapse of the slight concentration abnormality determination time duration since the instruction time point in the increase control, a difference is made between the NOx conversion rates in the case of severe concentration abnormality and in the case of gain deviation of the second NOx sensor 12. Accordingly, the instruction is given to make the supply amount of the reducing agent equal to the criterion supply amount. After conclusion of step S102, the flow goes to step S401.

At step S401, the ECU 10 determines whether the slight concentration abnormality determination time duration has elapsed since the start of the increase control at step S102. More specifically, it is determined whether a time duration that causes a distinguishable difference between the NOx conversion rates in the case of severe concentration abnormality and in the case of gain deviation of the second NOx sensor 12 has elapsed. In the case of an affirmative answer at step S401, the flow goes to step S1101. In the case of a negative answer at step S401, on the other hand, the flow repeats step S401.

At step S1101, the ECU 10 determines whether the NOx conversion rate at the current moment is decreased from the NOx conversion rate in the regular control. In the case of gain deviation of the second NOx sensor 12, the NOx conversion rate is decreased with an increase in supply amount of the reducing agent. In the case of severe concentration abnormality, on the other hand, the NOx conversion rate is increased with an increase in supply amount of the reducing agent. Accordingly, comparison between the NOx conversion rate in the regular control and the NOx conversion rate after elapse of the slight abnormality determination time duration in the increase control identifies which of gain deviation of the second NOx sensor 12 and severe concentration abnormality.

The determination of step S1101 may be replaced by determination of whether the NOx conversion rate at the current moment is lower than the sensor threshold. After elapse of the slight concentration abnormality determination time duration, the NOx conversion rate is equal to or higher than the sensor threshold in the case of severe concentration abnormality, while being lower than the sensor threshold in the case of gain deviation of the second NOx sensor 12. Accordingly, such determination also identifies which of severe concentration abnormality and gain deviation of the second NOx sensor 12.

In the case of an affirmative answer at step S1101, the flow goes to step S1002 to determine that the second NOx sensor 12 has gain deviation. In the case of a negative answer at step S1101, on the other hand, the flow goes to step S503 to determine that severe concentration abnormality occurs. The flow subsequently goes to step S104 to terminate the increase control.

As described above, this embodiment enables gain deviation of the second NOx sensor 12 to be detected with high accuracy. The second NOx sensor 12 may show a lower gain than the actual value. This results in increasing the calculated NOx conversion rate and makes it difficult to identify the type of abnormality described in this application. Such case is beyond the scope of this application.

1. A failure determination device for an emission control apparatus of an internal combustion engine, the failure determination device comprising:
a first supply valve that is provided in an exhaust conduit of the internal combustion engine to supply a reducing agent into the exhaust conduit;
a first selective reduction NOx catalyst that is provided downstream of the first supply valve in the exhaust conduit to selectively reduce NOx with the reducing agent adsorbed to the first selective reduction NOx catalyst;
a second supply valve that is provided downstream of the first selective reduction NOx catalyst in the exhaust conduit to supply the reducing agent into the exhaust conduit;
a second selective reduction NOx catalyst that is provided downstream of the second supply valve in the exhaust conduit to reduce NOx with the reducing agent adsorbed to the second selective reduction NOx catalyst;
a NOx sensor that is configured to detect a NOx concentration in an exhaust emission flowing out of the second selective reduction NOx catalyst; and
a controller that is configured to determine a supply amount of the reducing agent based on an amount of NOx flowing into the first selective reduction NOx catalyst and give an identical instruction to operate the first supply valve and the second supply valve, wherein
the controller gives an instruction to the first supply valve and the second supply valve such as to make a supply amount of the reducing agent from the first supply valve and the second supply valve larger than the supply amount of the reducing agent determined based on the amount of NOx flowing into the first selective reduction NOx catalyst, and determines whether the first supply valve is abnormal or the second supply valve is abnormal, based on a NOx concentration detected by the NOx sensor after elapse of a first specified time duration since a certain instruction time point when the instruction is given.

2. The failure determination device for the emission control apparatus of the internal combustion engine according to claim 1,
wherein the controller determines abnormality of the second supply valve, when a NOx conversion rate calculated from the NOx concentration detected by the NOx sensor after elapse of the first specified time duration since the instruction time point is equal to or higher than a supply valve threshold, and
the controller determines abnormality of the first supply valve, when the NOx conversion rate is lower than the supply valve threshold.

3. The failure determination device for the emission control apparatus of the internal combustion engine according to claim 1,
wherein the controller determines occurrence of slight concentration abnormality that provides a low concentration of the reducing agent, when a NOx conversion rate calculated from the NOx concentration detected by the NOx sensor after elapse of a second specified time duration, which is a shorter time period than the first specified time duration, since the instruction time point is equal to or higher than a supply valve threshold, and
the controller determines abnormality of either one of the first supply valve and the second supply valve, when the NOx conversion rate is lower than the supply valve threshold.

4. The failure determination device for the emission control apparatus of the internal combustion engine according to claim 3,
wherein the controller determines occurrence of one of the slight concentration abnormality, abnormality of the first supply valve and abnormality of the second supply valve, when the NOx conversion rate calculated from the NOx concentration detected by the NOx sensor prior to the instruction time point is equal to or higher than a severe reducing agent abnormality threshold, which is a smaller threshold value than the supply valve threshold, and
the controller determines occurrence of severe reducing agent abnormality that provides a lower concentration of the reducing agent than the concentration provided by the slight concentration abnormality, when the NOx conversion rate is lower than the severe reducing agent abnormality threshold.

5. The failure determination device for the emission control apparatus of the internal combustion engine according to claim 1,
the failure determination device further comprising a reducing agent concentration sensor that is configured to detect a concentration of the reducing agent, wherein
the controller determines occurrence of severe supply valve deterioration that is deterioration of both the first supply valve and the second supply valve, when the concentration of the reducing agent detected by the reducing agent concentration sensor is normal and a NOx conversion rate calculated from the NOx concentration detected by the NOx sensor prior to the instruction time point is lower than a severe reducing agent abnormality threshold.

6. The failure determination device for the emission control apparatus of the internal combustion engine according to claim 5,
wherein the controller determines abnormality of either one of the first supply valve and the second supply valve, when the concentration of the reducing agent detected by the reducing agent concentration sensor is normal, the NOx conversion rate calculated from the NOx concentration detected by the NOx sensor prior to the instruction time point is equal to or higher than the severe reducing agent abnormality threshold, and the NOx conversion rate calculated from the NOx concentration detected by the NOx sensor after elapse of a third specified time duration, which is a shorter time period than the first specified time duration, since the instruction time point is lower than a supply valve threshold, and
the controller determines occurrence of slight deterioration that is deterioration of both the first supply valve and the second supply valve and has a lower degree of deterioration than the severe supply valve deterioration, when the NOx conversion rate is equal to or higher than the supply valve threshold.

7. The failure determination device for the emission control apparatus of the internal combustion engine according to claim 1,
wherein the controller determines abnormality of the NOx sensor, when the NOx conversion rate calculated from the NOx concentration detected by the NOx sensor after elapse of a fourth specified time duration since the instruction time point is lower than a sensor threshold, which is a smaller threshold value than a supply valve threshold, and
the controller determines abnormality of the first supply valve, when the NOx conversion rate is equal to or higher than the sensor threshold and is lower than the supply valve threshold.

8. The failure determination device for the emission control apparatus of the internal combustion engine according to claim 3,
wherein the controller determines abnormality of the NOx sensor, when the NOx conversion rate calculated from the NOx concentration detected by the NOx sensor prior to the instruction time point is higher than zero and is lower than a severe reducing agent abnormality threshold and the NOx conversion rate calculated from the NOx concentration detected by the NOx sensor after elapse of a fifth specified time duration, which is a shorter time period than the first specified time duration, since the instruction time point is decreased from the NOx conversion rate calculated from the NOx concentration detected by the NOx sensor prior to the instruction time point, and
the controller determines occurrence of severe reducing agent abnormality that provides a lower concentration of the reducing agent than the concentration provided by the slight concentration abnormality, when the NOx conversion rate calculated from the NOx concentration detected by the NOx sensor after elapse of the fifth specified time duration is increased from the NOx conversion rate calculated from the NOx concentration detected by the NOx sensor prior to the instruction time point.
